(19) United States
(12) Patent Application Publication
(10) Pub. No.: US 2008/0176884 A1
(43) Pub. Date: Jul. 24, 2008

(54) 7,8-Saturated-4,5-Epoxy-Morphinanium Analogs

(75) Inventors: Julio Perez, Tarrytown, NY (US); Amy Qi Han, Hockessin, DE (US); Yakov Rotshteyn, Monroe, NY (US)

Correspondence Address: KELLEY DRYE & WARREN LLP, 400 ALTLANTIC STREET , 13TH FLOOR, STAMFORD, CT, 06901, US

(73) Assignee: Progenics Pharmaceuticals, Inc., Tarrytown, NY (US)

(21) Appl. No.: 11/944,400

(22) Filed: Nov. 21, 2007

Related U.S. Application Data
(60) Provisional application No. 60/867,099, filed on Nov. 22, 2006.
(60) Provisional application No. 60/867,390, filed on Nov. 27, 2006.

(51) Int. Cl. A61K 31/485; C07D 489/08; A61P 43/00

(52) U.S. Cl. 514/282; 546/45

(57) ABSTRACT

Novel 7,8-saturated-4,5-epoxy-morphinanium analogs are disclosed. Pharmaceutical compositions containing the 7,8-saturated-4,5-epoxy-morphinanium analogs and methods of their pharmaceutical uses are also disclosed. The compounds disclosed are useful, inter alia, as modulators of opioid receptors.

This application claims the benefit of U.S. provisional application Ser. No. 60/867,099 filed on Nov. 22, 2006, and U.S. provisional application 60/867,390 filed on Nov. 27, 2006, which are both herein incorporated by reference in their entireties.

### BACKGROUND OF THE INVENTION

1. Field of the Invention

The present invention generally relates to novel 7,8-single-bond-4,5-epoxy-morphinanium analogs (hereinafter referenced to as “7,8-saturated-4,5-epoxy-morphinaniums”), including 7,8-dihydro-4,5-epoxy-morphinanium analogs, synthetic methods for their preparation, pharmaceutical preparations comprising the same, and methods for their use.

2. Description of the Related Art

Opioids are agents that bind to certain receptors, known as opioid receptors, principally found in the central nervous system and gastrointestinal tract. They typically exhibit opium or morphine-like properties. There are four broad classes of opioids: endogenous opioid peptides, produced in the body; opium alkaloids, such as morphine (the prototypical opioid) and codeine; semi-synthetic opioids such as heroin and oxycodone; and fully synthetic opioids such as pethidine and methadone that have structures unrelated to the opium alkaloids. Although used synonymously with the term “opiate,” an the term “opiate” is more correctly used to describe an opioid compound derived from a natural opium alkaloid or a semi-synthetic derivative of such derived compound. Among the chemical classes of opioids with morphine-like activity are the purified alkaloids of opium consisting of phenanthrenes (morphine and codeine both share the phenanthrene or morphinan ring structure) and benzylisoquinolines, semi-synthetic derivatives of morphine, phenylpiperidine derivatives, morphinan derivatives, benzomorphan derivatives, diphenyl-heptane derivatives, and propionanilide derivatives.

Opioid compound receptors have been classified into at least four major lasses: mu (mu-1, mu-2 and mu-3), kappa and delta. An additional opioid receptor has been identified. This receptor is the nociceptin receptor or ORL 1 receptor. Each of these classes of receptors is believed to be distributed throughout the CNS and the periphery. All of these receptors have been hypothesized to be G-protein coupled receptors acting on GABAergic neurotransmission. Agonistic activation of these receptors activates K⁺ currents which increases K⁺ efflux, i.e., hyperpolarization, thereby reducing voltage-gated Ca²⁺ entry. Hyperpolarization of membrane potential by K⁺ currents and inhibition of the Ca²⁺ influx prevents neurotransmitter release and pain transmission in varying neuronal pathways. The pharmacological response to an opioid depends upon the receptor(s) it binds, its affinity for the receptor, and whether the opioid compound binds in a agonistic or antagonistic fashion. Activation of one receptor versus another may result in a distinct pharmacodynamic profile. For example, activation of the mu-1 receptor by the opioid agonist morphine may lead to supraspinal analgesia, while respiratory depression and physical dependence may be mediated by activation of the mu-2 receptor and spinal analgesia by the activation of the kappa-receptor.

Opioid compounds can be divided broadly into agonists and antagonists. The term “agonist” refers to a signaling molecule which binds to a receptor, inducing a conformational change that produces a response. The term “antagonist” broadly refers to a drug which attenuates the effect of the agonist.

Opioid compounds fall on a sliding scale of efficacy from a full agonist to an antagonist. For example, several morphinan derivatives having various substituents on the nitrogen atom have been found to exhibit narcotic antagonist as well as narcotic analgesic activity. Such compounds are referred to as agonist-antagonists. Pachter and Matossian, U.S. Pat. No. 3,393,197, disclose N-substituted-14-hydroxydihydronormorphines, including the N-cyclobutylmethyl derivative, commonly called nalbuphine. Monkovik and Thomas, U.S. Pat. No. 3,775,414, disclose N-cyclobutylmethyl-3,14-dihydroxymorphinan, commonly called butorphanol. Bentley et al., U.S. Pat. No. 3,433,791, disclose 17-(cyclopropylmethyl)-.alpha.-(1,1-dimethylethyl)-4,5-epoxy-18,19-dihydro-3-hydroxy-6-methoxy-.alpha.-methyl-6,14-ethenomorphinan-7-methanol, commonly called buprenorphine.

Opioid agonists are clinically used for a number of indications, including to produce analgesia and anesthesia, to suppress coughs, to alleviate diarrhea, to ameliorate the anxiety due to a shortness of breadth (oxymorphone) and in the detoxification of an opioid antagonist overdose. Beyond their clinically useful effects, opioid agonists have also been reported to have a number of side effects, including constipation, dysphoria, respiratory depression, dizziness, nausea, dependency, and pruritus. Some of these side effects may be associated with activation of peripheral rather than central receptors. For example, the administration of mu opioid agonists may result in intestinal dysfunction, such as constipation, due to the receptors in the wall of the gut. It is not an uncommon problem for patients having received prolonged doses of opioids to suffer from a particularly troublesome condition known as ileus, that is an obstruction of the bowel or gut, especially the colon, due to disruption of normal coordinated movements of the gut.

WO 2004/029059 N-quaternary hydromorphone agonists wherein the nitrogen carries a methyl substitutent and a C₁-C₆ substituent. Such compounds are asserted to provide potent mu-agonist activity, but to not cross the blood-brain barrier, thereby reducing opioid agonist CNS side effects. Similarly, WO 2004/043964 discloses N-methyl quaternary derivatives of antagonistic morphinan alkaloids, naltrexone and naloxe as potent antagonists of the mu receptor, which because of their ionic charge do not traverse the blood brain barrier into the central nervous system, thereby not blocking the central pain relieving activity of agonistic opioids when the two are concomitantly administered exogenously, or the endogenous opioid compounds produced naturally.

Certain opioid analogs are known to act as opioid receptor binding antagonists, that is, the analogs bind to the opioid receptors and interfere with the expression of opioid activity at the receptor sites. Opioid antagonists reverse the major pharmacodynamic actions of the opioid narcotics, such as analgesia, sedation, respiratory depression and myosis. Antagonists generally may be segregated into two broad classes, “surmountable” or “insurmountable” (or “unsurmontable”), on the basis of being competitive or non-competitive.

Mu opioid receptors, which have been classified as a G-protein coupled receptors (GPCR), have been suggested to have a constitutively active state that may be presented by μ* (see, e.g., U.S. Pat. No. 6,007,986). With no prior drug exposure (naïve state) the activity of the μ* state is believed to be minimal. Compounds that exhibit antagonist activity at a particular GPCR having basal signaling activity, such as the mu-opioid receptor, have been classified as either neutral antagonists or inverse agonists based on the effect which they exhibit upon the basal signaling activity of the particular receptor for which they are a ligand following interaction. “Inverse antagonists,” are agents which block the effects of an agonist at the target receptor and also suppress spontaneous receptor activity. By “neutral antagonist,” it is meant the compound simply binds to the receptor without changing its activity. A null antagonist may bind selectively to the resting, drug-sensitive mu receptor state, or to the constitutively active mu receptor state, or to both states.

Still other N-substituted morphinan derivatives are pure narcotic antagonists with little or no agonist activity. Lewenstein, U.S. Pat. No. 3,254,088, discloses N-allyl-7,8-dihydro-14-hydroxynormorphinone, commonly known as naloxone. Pachter and Matossian, U.S. Pat. No. 3,332,950, disclose N-substituted-14-hydroxy-dihydronormorphinones including the N-cyclopropylmethyl analog, commonly known as naltrexone. Compounds of these two patents are narcotic antagonists.

Naloxone and naltrexone are practically pure opioid antagonists devoid of analgesic activity (Bulberg, H.; Dayton, H. B. Narcotic Antagonists; Braude, M. C., Harris, L. S.; May, E. L.: Smith, J. P.; Villarrela, J. E., Ed.; Raven: New York, 1974; pp. 33-43). Competitive antagonists, such as naloxone and naltrexone, bind to the opioid receptors with higher affinity than agonists but do not activate the receptors. This displaces the agonist, attenuating and/or reversing the agonist effects. This effectively blocks the receptor, preventing the body from making use of opioids and endorphins, proteins that naturally bind to the opioid receptors. On the other hand, nalorphine and nalbuphine, despite their potent mu-antagonistic activity, as indicated above, have been reported to possess analgesic activity of their own through agonism at the opioid κ-receptor (Casy, A. F., Parfitt, R. T., Opioid Analgesics, Chemistry and Receptors; Plenum: New York, 1986; Chapter 4, pp. 153-214).

For many years, physical dependence or drug addiction caused by opioids have been treated by drug withdrawal through the administration of opioid antagonistic drugs, such as naltrexone and naloxone. Such treatment protocols may entail the substitution of another drug such as methadone, buprenorphine, or methadyl acetate for the opioid. Opioid overdose can also be rapidly reversed with an opioid antagonist.

More recently, there have been attempts to selectively antagonize opioid-induced side effects via the use of receptor antagonists, such as naloxone or nalmephene. However, the success may be said to be limited because these compounds may also reverse analgesia and induce opioid withdrawal (Yuan, C.-S. et al., J. Pharm. Exp. Ther. 300: 118-123 (2002)). For example, naloxone and naltrexone have been implicated as being useful in the treatment of gastrointestinal tract dysmotility (see, e.g., U.S. Pat. No. 4,987,126 and Kreek, M. J. Schaefer, R. A., Hahn, E. F., Fishman, J. Lancet, 1983, 1, 8319, 261 which disclose naloxone and other morphinan-based opioid antagonists (i.e., naloxone, naltrexone) for the treatment of idiopathic gastrointestinal dysmotility). Naloxone has also been reported to effectively treat non-opioid induced bowel obstruction, implying that the drug may act directly on the GI tract or in the brain (See, e.g., Schang, J. C., Devroede, G., Am. J. Gastroenerol., 1985, 80, 6, 407), and implicated as a therapy for paralytic ileus (Mack, D. J. Fulton, J. D., Br. J. Surg., 1989, 76, 10, 1101). However, it is well known that activity of naloxone and naltrexone is not limited to peripheral systems and may interfere with the analgesic effects of opioid narcotics.

A number of side-effects produced by opioid agonists are believed to be of central origin. In order to avoid such side effects, peripheral opioid agonists and antagonists that do no cross the blood-brain barrier into the central nervous system have been proposed and developed.

The use of quarternized opioid antagonists for selectively blocking the constipating effects of narcotic antagonists have been suggested (See, U.S. Pat. No. 4,806,556, Col. 2, lines 51-53), including some peripheral mu-antagonists derived from the structure naltrexone (Botros, et al., J. Med. Chem. 1989, 32, 2068-2071). A number of peripheral opioid antagonists developed have been tested in relation to their usefulness in preventing gastrointestinal side effects of the opioids. For example, in U.S. Pat. No. 5,250,542, U.S. Pat. No. 5,434,171, U.S. Pat. No. 5,159,081, and U.S. Pat. No. 5,270,328, peripherally selective piperidine-N-alkylcarboxylate opioid antagonists are described as being useful in the treatment of idiopathic constipation, irritable bowel syndrome, and opioid-induced constipation, WO 2004/043964, discussed above, discloses n-methyl quaternary derivatives of naltrexone and naloxone as binding to peripheral receptors primarily located in the gastrointestinal tract, and thereby mitigating through their antagonist activity undesirable side effects of opiate therapy such as constipation and nausea. U.S. Pat. No. 4,176,186 describes quaternary derivatives of noroxymorphone (i.e., methylnaltrexone) that are said to prevent or relieve the intestinal immobility side effect of narcotic analgesics without reducing analgesic effectiveness. U.S. Pat. No. 5,972,954 describes the use of methylnaltrexone, enteric-coated methylnaltrexone, or other quaternary derivatives of noroxymorphone for preventing and/or treating opioid- and/or nonopioid-induced side effects associated with opioid administration, including the decrease in intestinal motility associated with opioid use.

There is a need for other opioid compounds that do not have appreciable central activities and yet modulate of 10K receptors, particularly mu-opioid receptors. There is a further need for opioid compounds that protect against peripheral opioid activity and/or allow for positive opioid effects, such as analgesia, while minimizing the peripheral side effects of opioid administration, or that act intestinally to minimize the adverse effects of opioid administration on intestinal homeostasis.

### SUMMARY OF THE INVENTION

There is provided herein novel 7,8-saturated-4,5-epoxy-morphinaniums compounds, and in particular 7,8-dihydro-4,5-epoxy-morphinaniums, which bind to the μ-opioid receptor. In one embodiment, the 7,8-saturated-4,5-epoxy-morphinan compounds have limited or no blood-brain barrier as such do not act centrally so as to cause significant central side effects.

In one embodiment of the invention are disclosed compounds having the formula I:

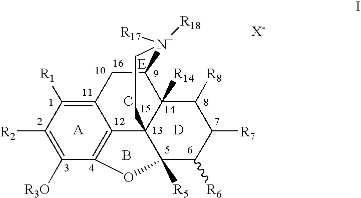

or pharmaceutically acceptable salt forms, polymorphs, or prodrugs thereof, wherein:
R₁₇ and R₁₈ are selected alternatively with respect to one another from (a) or (b):
   (a) unsubstituted or non-halogen substituted: C4-C20 (cycloalkyl)alkyl or (cycloalkenyl)alkyl, (cycloheteryl)alkyl, (cycloaryl)alkyl; C4-C10 (cycloalkyl)alkyl or (cycloalkenyl)alkyl, (cycloheteryl)alkyl, (cycloaryl)alkyl
   (b) substituted or unsubstituted linear or branched C1-C3 alkyl, C2-C3 alkenyl, or C3 alkynyl;
   wherein if (b) is selected as methyl, and R6 below is selected as ═O, (a) is not unsubstituted (cyclopropyl)methyl;
R is O, ═CH₂, —N(CH₃)₂, or any cyclic ring, or forms a cyclic ring with R₇;
R₇ and R₈ are H or alkyl;
R₁₄ is OH, halide, amido, amino, or forms a cyclic ring with R₁₈, and if R₆=a cyclic ring, or forms a cyclic ring with R₇, may further be an alkoxy or aryloxy, and if R₆ is not ═O, R₄ may be alkoxy or aryloxy;
R₁ and R₂ are independently H, halide, alkoxy, alkyl, or aryl
R₃ is H, C1-C4 alkyl, or C1-C3 acyl, -silyl;
R₅ is H, OH, alkyl, alkoxy, or aryloxy; and
X⁻ is an anion.

In another embodiment are disclosed compounds having the formula I(a):

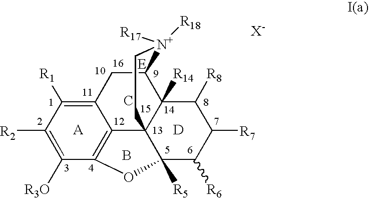

or pharmaceutically acceptable salt forms, polymorphs, or prodrugs thereof, wherein:
R₁ and R₂ are independently H, OH, OR₂₉, halide, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   or R₁ and R₂ are combined to form a C₃-C₆ carbocycle fused ring, a benzo fused ring,
   or a 5-6 membered heteroaryl fused ring;
R₃ is H, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₅ is H, OH, OR₂₉,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, N(CH₃)₂, or any cyclic ring;
R₇ is H, OH, OR₂₉,
   (C₁-C₂₀) alkyl substituted with 0-3 R₁₉;
   (C₂-C₂₀) alkenyl substituted with 0-3 R₁₉;
   (C₂-C₂₀) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   or R₆ and R₇ are combined to form an O-fused ring, a C₃-C₆ carbocycle fused ring, a benzo fused ring, or a 5-6 membered heteroaryl fused ring;
R₈ is H, OH, OR₂₉
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₁₄ is H, OH, halide,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀; aryloxy, acyloxy,
      or combined with R₁₈ to form an O-fused ring, or a C₃-C₆ carbocycle fused ring, or if R₆=a cyclic ring, or forms a cyclic ring with R₇, may be further be an alkoxy or aryloxy;
      wherein if R is ═O, R₁₄ is not:
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
R₁₇ is (C₄-C₂₀) alkyl substituted with 0-3 R₂₅;
   (C₄-C₂₀) alkenyl substituted with 0-3 R₂₅;
   (C₄-C₂₀) alkynyl substituted with 0-3 R₂₅;
   (C₃-C₁₀) cycloalkyl substituted with 0-3R₂₆;
   (C₃-C₁₀) carbocycle substituted with 0-3R₂₆;
   aryl substituted with 0-3R₂₆;
R₁₈ is (C₁-C₃) alkyl substituted with 0-3 R₂₇;
   (C₂-C₄) alkenyl substituted with 0-3 R₂₇;
   (C₂-C₄) alkynyl substituted with 0-3 R₂₇;
R₁₉ is at each occurrence is independently selected from:
   H, C₁-C₆ alkyl, CF₃, OR₂₄, Cl, F, Br, I, ═O, CN, NO₂, NR₂₂R₂₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, (C₁-C₆ alkyl)-C(═O)—, and (C₁-C₆ alkyl)-S(═O)₂—;
R₂₃, at each occurrence, is independently selected from:
   H, (C₁-C₆) alkyl,
   (C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅, at each occurrence, is independently selected from:
   H, C₁-C₆ alkyl, OR₂₄, ═O, CN, NO₂, NR₂₇R₂₈;
   C₃-C₁₀ carbocycle substituted with 0-3 R₂₇;
   aryl substituted with 0-3 R₂₇; or
   5 to 10 membered heterocycle containing 1 to 4 heteroatoms selected from nitrogen, oxygen, wherein said 5 to 10 membered heterocycle is substituted with 0-3 R₂₇;
R₂₆, at each occurrence, is independently selected from:
   H, (C₁-C₆)alkyl, benzyl, phenyl, phenethyl, (C₁-C₆ alkyl)-C(═O)—;
R₂₇, at each occurrence, is independently selected from:
   —OH, —OR₂₈, C₁-C₆ alkyl, C₁-C₄ alkoxy;
R₂₈, at each occurrence, is independently selected from:
   C₁-C₆ alkyl; (C₁-C₆ alkyl)-C(═O)—, and (C₁-C₆ alkyl)-S(═O)₂—; and
R₂₉ is at each occurrence is independently selected from:
   H, C₁-C₆ alkyl, CF₃, acyl(C₁-C₆)alkyl;
   acylaryl substituted with 0-3 R₂₁;
   C₃-C₁₀ carbocycle substituted with 0-3 R₂₁;
   aralkyl substituted with 0-3 R₂₁;
   5 to 10 membered heterocycle containing 1 to 4 heteroatoms selected from nitrogen, oxygen, and sulphur, wherein said 5 to 10 membered heterocycle is substituted with 0-3 R₂₁; or aryl substituted with 0-3R₂₀; and
   X⁻ is an anion

In yet another embodiment are disclosed compounds having the formula I(b):

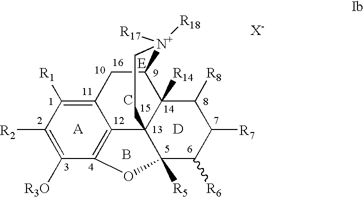

or pharmaceutically acceptable salt forms, polymorphs, or prodrugs thereof, wherein:
R₁ and R₂ are independently H, OH, OR₂₉, halide, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   or R₁ and R₂ are combined to form a C₃-C₆ carbocycle fused ring, a benzo fused ring,
   or a 5-6 membered heteroaryl fused ring;
R₃ is H, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₅ is H, OH, OR₂₉,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   amine, amide, sulfonamide, ester, heterocycle, cyclic carbohydride, aryl;
R₇ is H, OH, OR₂₉,
   (C₁-C₂₀) alkyl substituted with 0-3 R₁₉;
   (C₂-C₂₀) alkenyl substituted with 0-3 R₁₉;
   (C₂-C₂₀) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   or R₆ and R₇ are combined to form an O-fused ring, a C₃-C₆ carbocycle fused ring, a benzo fused ring, or a 5-6 membered heteroaryl fused ring;
R₅ is H, OH, OR₂₉
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₁₄ is H, OH,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀; aryloxy, acyloxy,
   or R₁₄ is combined with R₁₈ to form an O-fused ring, or a C₃-C₆ carbocycle fused ring;
   wherein if R₆═O, R₁₄ is not
      (C₁-C₈) alkyl substituted with 0-3 R₁₉;
      (C₂-C₈) alkenyl substituted with 0-3 R₁₉;
      (C₂-C₈) alkynyl substituted with 0-3 R₁₉;
      (C₃-C₁₀) cycloalkyl substituted with 0-3R₂₀;
      (C₃-C₁₀) carbocycle substituted with 0-3R₂₀;
R₁₇ is (C₄-C₁₀) alkyl substituted with 0-3 R₂₅;
   (C₄-C₁₀) alkenyl substituted with 0-3 R₂₅;
   (C₄-C₁₀) alkynyl substituted with 0-3 R₂₅;
   (C₃-C₁₀) cycloalkyl substituted with 0-3R₂₆;
   (C₃-C₁₀) carbocycle substituted with 0-3R₂₆;
   aryl substituted with 0-3R₂₆;
R₁₈ is (C₁-C₃) alkyl substituted with 0-3 R₂₇;
   (C₂-C₄) alkenyl substituted with 0-3 R₂₇;
   (C₂-C₄) alkynyl substituted with 0-3 R₂₇;
R₁₉ is at each occurrence is independently selected from:
   H, C₁-C₆ alkyl, CF₃, OR₂₄, Cl, F, Br, I, ═O, CN, NO₂, NR₂₂R₂₃;
   C₃-C₁₀ carbocycle substituted with 0-3 R₂₁;
   aryl substituted with 0-3 R₂₁; or
   a 5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, (C₁-C₆ alkyl)-C(═O)—, and (C₁-C₆ alkyl)-S(═O)₂—;
R₂₃, at each occurrence, is independently selected from:
   H, (C₁-C₆) alkyl,
   (C₁-C₆ alkyl)-C(═O)—, and (C₁-C₆ alkyl)-S(═O)₂—,
R₂₄, at each occurrence, is independently selected from H, phenyl, benzyl, (C₁-C₆) alkyl, and (C₂-C₆) alkoxyalkyl;
R₂₅, at each occurrence, is independently selected from:
   H, C₁-C₆ alkyl, OR₂₄, ═O, CN, NO₂, NR₂₇R₂₈;
   C₃-C₁₀ carbocycle substituted with 0-3 R₂₇;
   aryl substituted with 0-3 R₂₇; or
   a 5 to 10 membered heterocycle containing 1 to 4 heteroatoms selected from nitrogen, oxygen, wherein said 5 to 10 membered heterocycle is substituted with 0-3 R₂₇;
R₂₆, at each occurrence, is independently selected from: H, (C₁-C₆)alkyl, benzyl, phenyl, phenethyl, (C₁-C₆ alkyl)-C(═O)—;
R₂₇, at each occurrence, is independently selected from:
   —OH, —OR₂₈, C₁-C₆ alkyl, C₁-C₄ alkoxy;
R₂₈, at each occurrence, is independently selected from:
   C₁-C₆ alkyl; (C₁-C₆ alkyl)-C(═O)—, and (C₁-C₆ alkyl)-S(═O)₂—; and
R₂₉ is at each occurrence is independently selected from:
   H, C₁-C₆ alkyl, CF₃, acyl(C₁-C₆)alkyl;
   acylaryl substituted with 0-3 R₂₁;
   C₃-C₁₀ carbocycle substituted with 0-3 R₂₁;
   aralkyl substituted with 0-3 R₂₁;
   5 to 10 membered heterocycle containing 1 to 4 heteroatoms selected from nitrogen, oxygen, and sulphur, wherein said 5 to 10 membered heterocycle is substituted with 0-3 R₂₁; or aryl substituted with 0-3R₂₀; and
   X⁻ is an anion

In still another embodiment are disclosed compounds having the formula I(c):

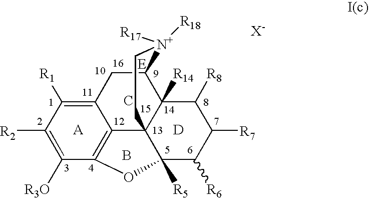

or pharmaceutically acceptable salt forms, polymorphs, or prodrugs thereof,
wherein:
R₁₇ and R₁₈ are selected alternatively with respect to one another from (a) or (b):
   (a) unsubstituted or non-halogen substituted: C₄-C₂₀ (cycloalkyl)alkyl or (cycloalkenyl)alkyl, (cycloheteryl)alkyl, (cycloaryl)alkyl; C₄-C₁₀ (cycloalkyl)alkyl or (cycloalkenyl)alkyl, (cycloheteryl)alkyl, (cycloaryl)alkyl
   (b) substituted or unsubstituted linear or branched C₁-C₃ alkyl, C₂-C₃ alkenyl, or C₃-alkynyl;
   wherein if (b) is selected as methyl and R6 below is selected ═O, (a) is not an unsubstituted (cyclopropyl)methyl;
R₆ is ═O, ═CH₂, —N(CH₃)₂, or any cyclic ring, or forms a cyclic ring with R₇;
R₇ and R₈ are H, hydrocarbyl, cyclohydrocarbyl, alkoxy, amine, amide, hydroxy or substituted moieties thereof;
R₁₄ is H. Off, halide, N-alkyl, N-dialkyl, N-aryl, N-alkylaryl, N-cycloalkylalkyl, or forms a cyclic ring with R₁₇ or R₁₈; and if R₆ is not ═O, R₁₄ may be alkoxy, aryloxy, or aryl-alkoxy;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

In another embodiment of formula Ic, are disclosed compounds, or pharmaceutically acceptable salt forms or prodrugs thereof, wherein, R₇ and R₈ are H or alkyl.

In yet another embodiment are disclosed compounds having the formula I(d):

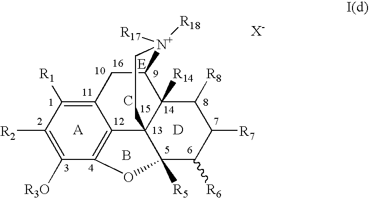

or pharmaceutically acceptable salt forms, polymorphs, or prodrugs thereof,
wherein:
R₁₇ and R₁₈ are a substituted or unsubstituted hydrocarbyl, when R₆ is ═O at least one of which is not methyl when the other is unsubstituted cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₈ are H or hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, SR₂₅, S(═O)R₂₅, SO₂R₂₅, or forms a cyclic ring with R₁₇ or R₁₈; and if R₆ is not ═O, R₁₄ may be alkoxy, aryloxy, or aryl-alkoxy;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.

Also disclosed are compounds having the formula I(e):

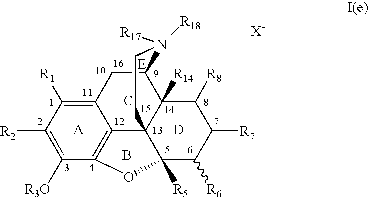

or a pharmaceutically acceptable salt form, polymorph, or prodrug thereof, wherein:
R₁ and R₂ are independently H, OH, OR₂₉, halide, silyl;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀);
   (C₃-C₁₀) carbocycle substituted with 0-3R₂₀;
   aryl substituted with 0-3R₂₀;
   or R₁ and R₂ are combined to form a C₃-C₆ carbocycle fused ring, a benzo fused ring,
   or a 5-6 membered heteroaryl fused ring;
R₃ is H, silyl, CO₂R₁₉, SO₂R₁₉, B(OR₁₉)₂;
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₅ is H, OH, OR₂₉,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₆ is H, ═O, N(CH₃)₂, ═(R₁₉)(R_{19′}), =(hetero cycle substituted with 0-3R₂₀), =(C₃-C₇ cycle substituted with 0-3R₂₀) or any cyclic ring;
R₇ is H, OH, OR₂₉,
   (C₁-C₂₀) alkyl substituted with 0-3 R₁₉;
   (C₂-C₂₀) alkenyl substituted with 0-3 R₁₉;
   (C₂-C₂₀) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
   or R₆ and R₇ are combined to form an O-fused ring, a C₃-C₆ carbocycle fused ring, a benzo fused ring, 5-, 6- or a 5-6 membered aryl with 0-3 R₂₀, or a heteroaryl fused ring;
R₈ is H, OH, OR₂₉, hetero cycle with 0-3R₂₀, alkylaryl with 0-3R₂₀, arylalkyl with 0-3 R₂₀,

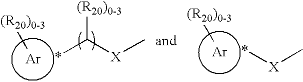

wherein, X is bond, ═O, O, S, N(R₂₉), SO, SO₂, SO₂N(R₂₉), CON(R₂₉), N(R₂₉)CON(R₂₉), N(R₂₉)C(═NR_{29′})N(R_{29″}), COO,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀;
R₁₄ is H, OH, halide, hetero cycle with 0-3R₂₀, alkylaryl with 0-3R₂₀, arylalkyl with 0-3 R₂₀,

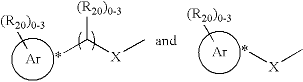

wherein, X is bond, ═O, O, S, N(R₂₉), SO, SO₂, SO₂N(R₂₉), CON(R₂₉), N(R₂₉)CON(R_{29′}), N(R₂₉)C(═NR_{29′})N(R_{29″}), COO,
   (C₁-C₈) alkyl substituted with 0-3 R₁₉;
   (C₂-C₈) alkenyl substituted with 0-3 R₁₉;
   (C₂-C₈) alkynyl substituted with 0-3 R₁₉;
   (C₃-C₁₀) cycloalkyl substituted with 0-3R₂₀;
   (C₃-C₁₀) carbocycle substituted with 0-3R₂₀;
   aryl substituted with 0-3R₂₀; aryloxy, acyloxy,
or combined with R₁₈ to form an O-fused ring, or a C₃-C₆ carbocycle fused ring, or if R₆=a cyclic ring, or forms a cyclic ring with R₇, may be further be an alkoxy or aryloxy;
   wherein if R₆ is ═O, R₁₄ is not:
      (C₁-C₈) alkyl substituted with 0-3 R₁₉;
      (C₂-C₈) alkenyl substituted with 0-3 R₁₉;
      (C₂-C₈) alkynyl substituted with 0-3 R₁₉;
      (C₃-C₁₀) cycloalkyl substituted with 0-3R₂₀;
      (C₃-C₁₀) carbocycle substituted with 0-3R₂₀;
R₁₇ is hetero cycle with 0-3R₂₀, alkylaryl with 0-3R₂₀, arylalkyl with 0-3 R₂₀,

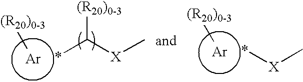

wherein, X is bond, ═O, O, S, N(R₂₉), SO, SO₂, SO₂N(R₂₉), CON(R₂₉), N(R₂₉)CON(R_{29′}), N(R₂₉)C(═NR_{29′})N(R_{29″}), COO,
   (C₄-C₂₀) alkyl substituted with 0-3 R₂₅;
   (C₄-C₂₀) alkenyl substituted with 0-3 R₂₅;
   (C₄-C₂₀) alkynyl substituted with 0-3 R₂₅;
   (C₃-C₁₀) cycloalkyl substituted with 0-3R₂₆;
   (C₃-C₁₀) carbocycle substituted with 0-3R₂₆;
   aryl substituted with 0-3R₂₆;
R₁₈ is (C₁-C₃) alkyl substituted with 0-3 R₂₇;
   (C₂-C₄) alkenyl substituted with 0-3 R₂₇;
   (C₂-C₄) alkynyl substituted with 0-3 R₂₇;
R₁₉ is at each occurrence is independently selected from:
   H, aryl substituted with 0-3R₂₀, C₁-C₆ alkyl, CF₃, OR₂₄, Cl, F, Br, I, ═O, CN, NO₂, NR₂₂R₂₃;
   C₃-C₁₀ carbocycle substituted with 0-3 R₂₁;
   aryl substituted with 0-3 R₂₁; or
   5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl, OR₂₅, XR₂₅,
   C₁-C₆ alkyl, C₁-C₄ alkoxy, C₁-C₄ haloalkyl,
   C₁-C₄ haloalkoxy, and C₁-C₄ haloalkyl-S—; or
   NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, hetero aryl, hetero cycle, alkylaryl, arylalkyl,
   (C₁-C₆ alkyl)-C(═O)—, and (C₁-C₆ alkyl)-S(═O)₂—;
R₂₃, at each occurrence, is independently selected from:
   H, (C₁-C₆) alkyl, C₆-C₁₀ aryl, hetero aryl, hetero cycle, alkylaryl, haloalkyl, and arylalkyl,
   (C₁-C₆ alkyl)-C(═O)—, and (C₁-C₆ alkyl)-S(═O)₂—;
   wherein R₂₂ and R₂₃ may further be combined to form 5-, 6-, 5-6-membered cycle with 0-3R₂₀;
R₂₄, at each occurrence, is independently selected from H, phenyl, benzyl, (C₁-C₆) alkyl, haloalkyl and (C₂-C₆) alkoxyalkyl;
R₂₅, at each occurrence, is independently selected from:
   H, C₁-C₆ alkyl, haloalkyl, OR₂₄, ═O, CN, NO₂, NR₂₇R₂₈;
   C₃-C₁₀ carbocycle substituted with 0-3 R₂₇;
   aryl substituted with 0-3 R₂₇; or
   5 to 10 membered heterocycle containing 1 to 4 heteroatoms selected from nitrogen, oxygen, wherein said 5 to 10 membered heterocycle is substituted with 0-3 R₂₇;
R₂₆, at each occurrence, is independently selected from:
   H, (C₁-C₆)alkyl, benzyl, phenyl, phenethyl, (C₁-C₆ alkyl)-C(═O)—;
R₂₇, at each occurrence, is independently selected from:
   —OH, —OR₂₈, C₁-C₆ alkyl, C₁-C₄ alkoxy;
R₂₈, at each occurrence, is independently selected from:
   C₁-C₆ alkyl; (C₁-C₆ alkyl)-C(═O)—, and (C₁-C₆ alkyl)-S(═O)₂—; and
R₂₉ is at each occurrence is independently selected from:
   H, C₁-C₆ alkyl, CF₃, acyl(C₁-C₆)alkyl;
   acylaryl substituted with 0-3 R₂;
   C₃-C₁₀ carbocycle substituted with 0-3 R₂₁;
   aralkyl substituted with 0-3 R₂₁;
   5 to 10 membered heterocycle containing 1 to 4 heteroatoms selected from nitrogen, oxygen, and sulphur, wherein said 5 to 10 membered heterocycle is substituted with 0-3 R₂; or aryl substituted with 0-3R₂₀; and
   X⁻ is an anion.

Also included as useful for the conditions discussed herein are the prodrugs, pharmaceutically acceptable salts, stereoisomers, polymorphs, hydrates, solvates, acid hydrates and N-oxides of the compounds of formula I, I(a), I(b), I(c), I(d) and I(e). For example, prodrugs are known to enhance a number of desirable pharmaceutical qualities (e.g., solubility, bioavailability, manufacturing, etc.). Prodrugs of the compounds of formula I, I(a), I(b), I(c), I(d) and I(e) may be prepared by modifying functional groups present in the compound in such a way that the modifications are cleaved, either in routine manipulation or in vivo, to the parent compound.

Further provided herein is a composition of matter selected from a salt, polymorph, or prodrug of one or more of the group consisting of:
17-cyclopropylmethyl-4,5α-epoxy-3,14-dihydroxy-17-methyl-6-methylenemorphinanium;
17-cyclopropylmethyl-4,5α-epoxy-14-hydroxy-17-methyl-3-propyloxy-6-oxomorphinanium;
17-Allyl-17-cyclopropylmethyl-4,5α-epoxy-3,14-dihydroxy-6-oxomorphinanium;
17-cyclobutylmethyl-4,5α-epoxy-3,14-dihydroxy-17-methyl-6-oxomorphinanium;
17-cyclopentylmethyl-4,5α-epoxy-3,14-dihydroxy-17-methyl-6-methylenemorphinanium;
17-(3,3′-dimethylallyl)-4,5α-epoxy-3,14-dihydroxy-17-methyl-6-oxomorphinanium;
17-(3′-phenylbut-2′-ynyl)-4,5α-epoxy-3,14-dihydroxy-17-methyl-6-oxomorphinanium;
17-(2′,2′-Difluorocyclopropyl)methyl-4,5α-epoxy-3,14-dihydroxy-17-methyl-6-oxomorphinanium;
17-cyclopropylmethyl-4,5α-epoxy-3-benzyloxy-14-hydroxy-17-methyl-6α-methoxy-morphinanium; and
17-cyclopropylmethyl-4,5α-epoxy-3,14-dihydroxy-17-methyl-6β-hydroxy-8-propoxy-morphinanium;
17-(2′-Methylcyclopropyl)methyl-4,5α-epoxy-3,14-dihydroxy-17-methyl-6-oxomorphinanium;
17-Cyclopropylmethyl-4,5α-epoxy-3,14-dihydroxy-17-methyl-6α-methoxy morphinanium;
17-Cyclopropylmethyl-4,5α-epoxy-3,14-dihydroxy-17-methyl-6β-methoxy morphinanium;
17-Cyclopropylmethyl-4,5α-epoxy-3-methoxy-14-hydroxy-17-methyl-6-methylenemorphinanium;
17-Cyclopropylmethyl-4,5α-epoxy-3,14-dihydroxy-17-methylmorphinanium;
3-Acetyl-17-cyclopropylmethyl-4,5α-epoxy-14-hydroxy-17-methylmorphinanium;
17-[(2′-tetrahydrofuryl)methyl]-4,5α-epoxy-3,14-dihydroxy-17-methyl-6-oxo-morphinaninium;
17-Cyclopropylmethyl-4,5α-epoxy-3-hydroxy-17-methyl-14-(3′-phenylpropyloxy)morphinanium;
17-Cyclopropylmethyl-4,5α-epoxy-3-hydroxy-17-methyl-14-propyloxy morphinanium;
17-Cyclopropylmethyl-4,5α-epoxy-3-hydroxy-17-methyl-14-methoxy-morphinanium;
17-methyl-4,5α-epoxy-3-hydroxy-(17,14-N,O-ethylene-6-oxo-morphinanium; and
17-Cyclopropylmethyl-4,5α-epoxy-3-hydroxy-(17,14-N,O-ethylene)-6-oxo-morphinanium.

### DETAILED DESCRIPTION OF THE INVENTION

There is still an need for compounds that may be used in methods to agonize or antagonize opioid receptors, particularly for use in preventing or treating the undesirable side effects associated with a administering exogenous opioids. The present disclosure is directed to these desired compounds, as well as other important ends.

In embodiments of the present invention, there are disclosed compounds useful in methods to agonize or antagonize opioid receptors, particularly the mu-opioid receptor. Of particular interest are compounds that act peripherally not passing through the blood-brain barrier.

Opioid receptor binding activity may be adjudged using a receptor binding assay well known in the art. For example, a radioligand dose-displacement assay may be run using diprenorphine as the agent to be displaced. An unlabeled opioid antagonist, such as naloxone, can serve as a positive control. The assay may be performed in a well array with binding reactions terminated by rapid filtration and harvesting with a harvester.

Generally there are disclosed compounds of formula I, I(a), I(b), I(c), I(d), I(e) set forth above in the “Summary of Invention.”

In particular embodiments, there is disclosed a group of μ-receptor binding 7,8-saturated-4,5-epoxy-morphinaniums having the structures of:

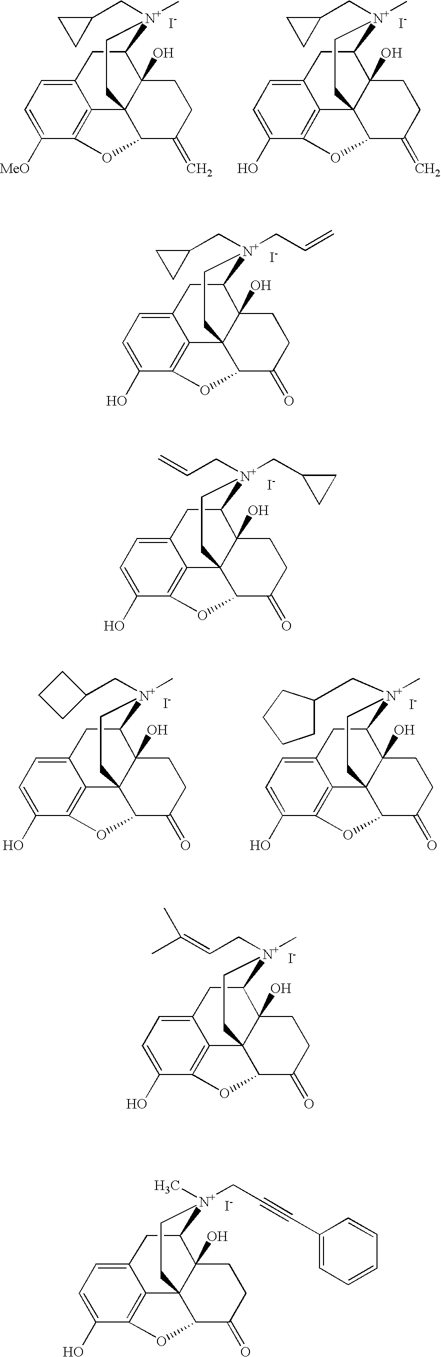

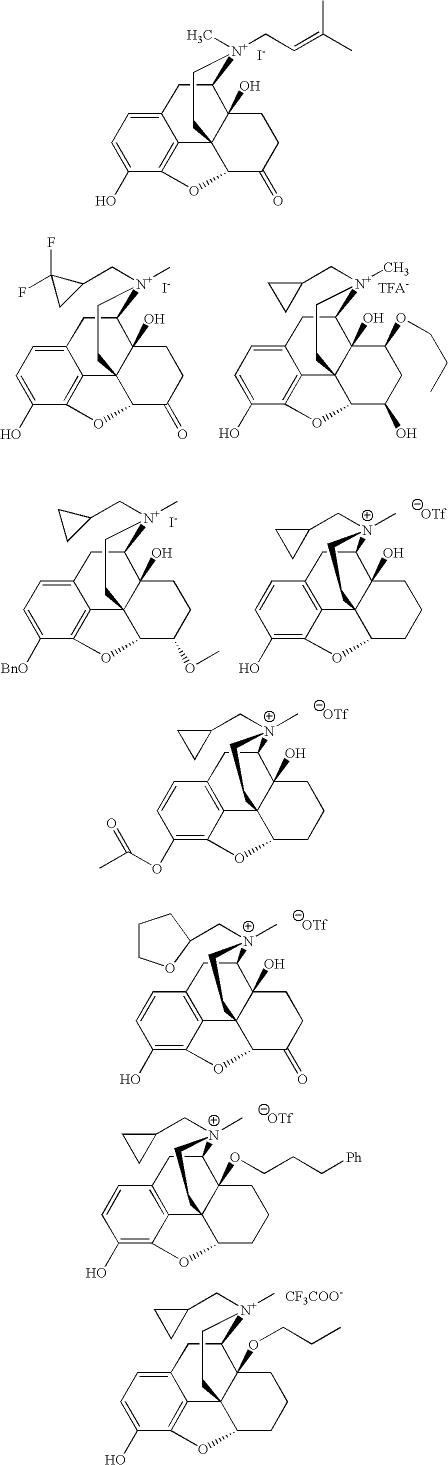

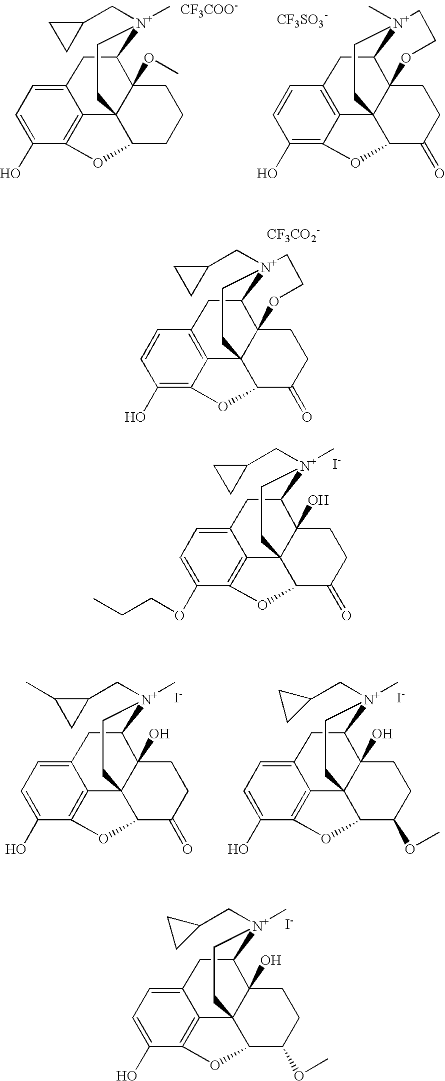

The term “acyl”, whether used alone, or within a term such as “acylamino”, denotes a radical provided by the residue after removal of hydroxyl from an organic acid. The term “acylamino” embraces an amine radical substituted with an acyl group. An examples of an “acylamino” radical is acetylamine (CH₃C(═O)—NH—). The term “aryloxy” denotes a radical provided by the residue after removal of hydrido from a hydroxy-substituted aryl moiety (e.g., phenol).

As used herein, “alkanoyl” refers to a-C(═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

The term “alkenyl” includes unsaturated aliphatic groups analogous in length and possible substitution to the alkyls described above, but that contain at least one double bond and must contain at least two carbon atoms. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “lower alkylene” herein refers to those alkylene groups having from about 1 to about 6 carbon atoms. The term “alkenyl” includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkenylene”, in general, refers to an alkylene group containing at least one carbon-carbon double bond. Exemplary alkenylene groups include, for example, ethenylene (—CH═CH—) and propenylene (—CH═CHCH₂—). Preferred alkenylene groups have from 2 to about 4 carbons.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro chloro or bromo to provide “haloalkoxy” or “haloalkoxyalkyl” radicals. Examples of “alkoxy” radicals include methoxy butoxy and trifluoromethoxy.

“Alkyl” in general, refers to an aliphatic hydrocarbon group which may be straight, branched or cyclic having from 1 to about 10 carbon atoms in the chain, and all combinations and subcombinations of ranges therein, e.g., a cycloalkyl, branched cycloalkylalkyl, a branched alkylcycloalkyl having 4-10 carbon atoms. The term “alkyl” includes both “unsubstituted alkyls” and “substituted alkyls,” the latter of which refers to alkyl moieties having substituents replacing a hydrogen on one or more carbons of the backbone. “Lower alkyl” refers to an alkyl group having 1 to about 6 carbon atoms. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2-dimethylbutyl, and 2,3-dimethylbutyl, cyclopropylmethyl and cyclobutylmethyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. The term “aralkyl” embraces aryl-substituted alkyl radicals such as benzyl, diphenylmethyl, triphenylmethyl, phenethyl, phenylpropyl, and diphenethyl. The terms benzyl and phenylmethyl are interchangeable. The term “n-alkyl” means a straight chain (i.e. unbranched) unsubstituted alkyl group. “Branched” refers to an alkyl group in which a lower alkyl group, such as methyl, ethyl or propyl, is attached to a linear alkyl chain.

An “alkylating agent” is a compound that can be reacted with a starting material to bind, typically covalently, an alkyl group to the starting material. The alkylating agent typically includes a leaving group that is separated from the alkyl group at the time of attachment to the starting material. Leaving groups may be, for example, halogens, halogenated sulfonates or halogenated acetates. An example of an alkylating agent is cyclopropylmethyl iodide.

The term “alkylsilyl” denotes a silyl radical substituted with an alkyl group. The term “alkylsilyloxy” denotes a silyloxy radical (—O—Si—) substituted with an alkyl group. An example of an “alkylsilyloxy” radical is —O—Si-t-BuMe₂.

The term “alkylsulfinyl” embraces radicals containing a linear or branched alkyl radical, of one to ten carbon atoms, attached to a divalent —S(═O)— atom. The term “arylsulfinyl” embraces aryl radicals attached to a divalent —S(═O)— atom (e.g., —S═OAr).

The term “alkylthio” embraces radicals containing a linear or branched alkyl radical, of one to ten carbon atoms, attached to a divalent sulfur atom. The term “arylsulfenyl” embraces aryl radicals attached to a divalent sulfur atom (—SAr) An example of “alkylthio” is methylthio, (CH₃—(S)—).

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups.

The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkyl radicals substituted with an N-alkyl-N-hydroxyamido radical. The term “amidoalkyl” embraces alkyl radicals substituted with amido radicals.

The term “aminoalkyl” embraces alkyl radicals substituted with amine radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl.

“Aryl-substituted alkyl”, in general, refers to an linear alkyl group, preferably a lower alkyl group, substituted at a carbon with an optionally substituted aryl group, preferably an optionally substituted phenyl ring. Exemplary aryl-substituted alkyl groups include, for example, phenylmethyl, phenylethyl and 3-(4-methylphenyl)propyl.

The term “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred “carbocycle” are cyclopropyl, cyclobutyl, cyclopentyl, and cyclohexyl.

The term “cycloalkyl” embraces radicals having three to ten carbon atoms, such as cyclopropyl cyclobutyl, cyclopentyl, cyclohexyl and cycloheptyl.

“Cycloalkyl-substituted alkyl”, in general, refers to a linear alkyl group, preferably a lower alkyl group, substituted at a terminal carbon with a cycloalkyl group, preferably a C₃-C₈ cycloalkyl group. Typical cycloalkyl-substituted alkyl groups include cyclohexylmethyl, cyclohexylethyl, cyclopentylethyl, cyclopentylpropyl, cyclopropylmethyl and the like.

“Cycloalkenyl”, in general, refers to an olefinically unsaturated cycloalkyl group having from about 4 to about 10 carbons, and all combinations and subcombinations of ranges therein. In some embodiments, the cycloalkenyl group is a C₅-C₈ cycloalkenyl group, i.e., a cycloalkenyl group having from about 5 to about 8 carbons.

“Dipolar aprotic” solvents are protophilic solvents that cannot donate labile hydrogen atoms and that exhibit a permanent dipole moment. Examples include acetone, ethyl acetate, dimethyl sulfoxide (DMSO), dimethyl formamide (DMF) and N-methylpyrrolidone.

“Dipolar protic” solvents are those that can donate labile hydrogen atoms and that exhibit a permanent dipole moment. Examples include water, alcohols such as 2-propanol, ethanol, methanol, carboxylic acids such as formic acid, acetic acid, and propionic acid.

The phrase “does not substantially cross,” as used herein, means that less than about 20% by weight of the compound employed in the present methods crosses the bloodbrain barrier, preferably less than about 15% by weight, more preferably less than about 10% by weight, even more preferably less than about 5% by weight and most preferably 0% by weight of the compound crosses the blood-brain barrier.

The term “halo” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals.

As used herein, the term “heterocycle” or “heterocyclic ring” is intended to mean a stable 5- to 7-membered monocyclic or bicyclic or 7- to 14-membered bicyclic heterocyclic ring which is saturated, partially unsaturated, or unsaturated (aromatic), and which consists of carbon atoms and 1, 2, 3 or 4 heteroatoms independently selected from the group consisting of N, O and S and including any bicyclic group in which any of the above-defined heterocyclic rings is fused to a benzene ring. Examples of saturated heterocyclic radicals include pyrrolidyl and morpholinyl.

The term “hydroxyalkyl” embraces linear or branched alkyl radicals having one to about ten carbon atoms any one of which may be substituted with one or more hydroxyl radicals.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

The terms “N-alkylamino” and “N,N-dialkylamino” denote amine groups which have been substituted with one alkyl radical and with two alkyl radicals, respectively.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

“Organic solvent” has its common ordinary meaning to those of skill in this art. Exemplary organic solvents useful in the invention include, but are not limited to tetrahydrofuran, acetone, hexane, ether, chloroform, acetic acid, acetonitrile, chloroform, cyclohexane, methanol, and toluene. Anhydrous organic solvents are included.

As used herein, “patient” refers to animals, including mammals, preferably humans.

As used herein, “peripheral” or “peripherally-acting” refers to an agent that acts outside of the central nervous system. As used herein, “centrally-acting” refers to an agent that acts within the central nervous system (CNS). The term “peripheral” designates that the compound acts primarily on physiological systems and components external to the central nervous system. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably 0% of the pharmacological activity of the compounds employed in the present methods is exhibited in the CNS.

It should also be understood that when referring to compounds of the invention, it is meant to encompass hydrates, solvates, and polymorphs of the same. Hydrates are formed when water binds to the crystal structure of a compound in a fixed stoichiometric ratio, although generally this ratio will change depending on the surrounding humidity with which the hydrate is in equilibrium. Hydration is a more specific form of salvation. Solvates are crystalline solid adducts containing either stoichiometric or nonstoichiometric amounts of a solvent incorporated within the crystal structure. If the incorporated solvent is water, the solvates are also commonly known as hydrates. Hydrates and solvates are well known to those or ordinary skill in the art.

Pharmaceutical polymorphism is characterized as the ability of a drug substance to exist as two or more crystalline phases that have different arrangements and/or conformations of the molecules in the crystal lattice. Amorphous solids consist of disordered arrangements of molecules and do not possess a distinguishable crystal lattice. Polymorphism refers to the occurrence of different crystalline forms of the same drug substance. Polymorphs are well know to those of ordinary skill in the art.

Polymorphs or solvates of a pharmaceutical solid can have different chemical and physical properties such as melting point, chemical reactivity, apparent solubility, dissolution rate, optical and electrical properties, vapor pressure, and density. These properties can have a direct impact on the processing of drug substances and the quality or performance of drug products. Chemical and physical stability, dissolution, and bioavailability are some of these qualities. A metastable pharmaceutical solid form may change crystalline structure or solvate or desolvate in response to changes in environmental conditions, processing, or over time. New, previously unknown polymorphs can develop spontaneously and unpredictably over time.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic-, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine. Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, the term “side effect” refers to a consequence other than the one (s) for which an agent or measure is used, as the adverse effects produced by a drug, especially on a tissue or organ system other then the one sought to be benefited by its administration.

As used herein, “stereoisomers” refers to compounds that have identical chemical constitution, but differ as regards the arrangement of the atoms or groups in space.

The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂ NH₂). The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical.

“Tertiary amines” has its common, ordinary meaning. In general, the tertiary amines useful in the invention have the general formula:

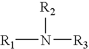

wherein R₁, R₂, and R₃ are identical or a combination of different straight or branched chain alkyl groups, alkenyl groups, alkylene groups, alkenylene groups, cycloalkyl groups, cycloalkyl-substituted alkyl groups, cycloalkenyl groups, alkoxy groups, alkoxy-alkyl groups, acyl groups, aryl groups, aryl-substituted alkyl groups, and heterocyclic groups. Exemplary tertiary amines useful according to the invention are those where R₁₋₃ is an alkyl group of the formula (CₙH₂ₙ₊₁, n=1-4), or aralkyl group of the formula (C₆H₅(CH₂)ₙ— [n=1-2]. Exemplary tertiary amines useful according to the invention also are cycloalkyl tertiary amines (e.g., N-methylmorpholine, N-methylpyrrolidine, N-methylpiperidine), pyridine and Proton Sponge® (N,N,N′,N′-tetramethyl-1,8-naphthalene).

The subjects to which the compounds of the present invention may be administered are vertebrates, in particular mammals. In one embodiment the mammal is a human, nonhuman primate, dog, cat, sheep, goat, horse, cow, pig and rodent. In one embodiment, the mammal is a human.

The pharmaceutical preparations of the invention, when used alone or in cocktails, are administered in therapeutically effective amounts. A therapeutically effective amount will be determined by the parameters discussed below; but, in any event, is that amount which establishes a level of the drug(s) effective for treating a subject, such as a human subject, having one of the conditions described herein. An effective amount means that amount alone or with multiple doses, necessary to delay the onset of, lessen the severity of, or inhibit completely, lessen the progression of, or halt altogether the onset or progression of the condition being treated or a symptom associated therewith.

An effective amount of a pharmaceutical preparation of the invention having primarily opioid agonist activity, in particular, mu-opioid agonist activity, is an amount that prevents, treats, or manages at least one symptom of acute or chronic pain, hyperalgesia, diarrhea, or anxiety due to shortness of breath. The effective amount is an amount that reduces coughing in one case. The effective amount of the opioid agonist may provide sedation or anesthesia.

The art defines constipation as (i) less than one bowel movement in the previous three days or (ii) less than three bowel movements in the previous week (see e.g, U.S. Pat. No. 6,559,158). In the case of constipation, an effective amount of an opioid antagonist, for example, is that amount which relieves a symptom of constipation, which induces a bowel movement, which increases the frequency of bowel movements, or which decreases oral-cecal transit time. Effective amounts therefore can be those amounts necessary to establish or maintain regular bowel movements.

Patients using opioids chronically include late stage cancer patients, elderly patients with osteoarthritic changes, methadone maintenance patients, neuropathic pain and chronic back pain patients. Treatment of these patients is important from a quality of life standpoint, as well as to reduce complications arising from chronic constipation, such as hemorrhoids, appetite suppression, mucosal breakdown, sepsis, colon cancer risk, and myocardial infarction.

Patients receiving treatment using the compounds of the present invention may concurrently or sequentially be receiving opioids. Compounds disclosed herein may be mixed with a conventional opioid compound. Conventional opioids include those selected from the group consisting of alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sulfentanil, tilidine, trimebutine, and tramadol. Optionally, an non-opioid anesthetic/antipyretic such as acetaminophen may be admixed with the opioid, in particular with oxycodone. The opioid also may be moved together with the compounds disclosed herein and provided in any of the forms described herein.

Dosage may be adjusted appropriately to achieve desired drug levels, local or systemic, depending on the mode of administration. For example, it is expected that the dosage for oral administration of the opioid in an enterically-coated formulation would be lower than in an immediate release oral formulation. In the event that the response in a patient is insufficient at such doses, even higher doses (or effectively higher dosage by a different, more localized delivery route) may be employed to the extent that the patient tolerance permits. Multiple doses per day are contemplated to achieve appropriate systemic levels of compounds. Appropriate systemic levels can be determined by, for example, measurement of the patient's peak or sustained plasma level of the drug. “Dose” and “dosage” are used interchangeably herein.

A variety of administration routes are available. The particular mode selected will depend, of course, upon the particular combination of drugs selected, the severity of the condition being treated, or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, transdermal, sublingual, intravenous infusion, pulmonary, intra-arterial, intra-adipose tissue, intra-lymphatic, intramuscular, intracavity, aerosol, aural (e.g., via cardrops), intranasal, inhalation, intra-articular, needleless injection, subcutaneous or intradermal (e.g., transdermal) delivery. For continuous infusion, a patient-controlled analgesia (PCA) device or an implantable drug delivery device may be employed. Oral, rectal, or topical administration may be important for prophylactic or long-term treatment. Preferred rectal modes of delivery include administration as a suppository or enema wash.

The pharmaceutical preparations may conveniently be presented in unit dosage form and may be prepared by any of the methods well known in the art of pharmacy. All methods include the step of bringing the compounds of the invention into association with a carrier which constitutes one or more accessory ingredients. In general, the compositions are prepared by uniformly and intimately bringing the compounds of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

It should be understood that when referring to compounds of the invention, it is meant to encompass salts of the same. Such salts are of a variety well known to those or ordinary skill in the art. When used in pharmaceutical preparations, the salts preferably are pharmaceutically-acceptable for use in humans. Bromide is an example of one such salt.

The pharmaceutical preparations of the present invention may include or be diluted into a pharmaceutically-acceptable carrier. The term “pharmaceutically-acceptable carrier” as used herein means one or more compatible solid or liquid fillers, diluents or encapsulating substances which are suitable for administration to a human or other mammal such as non-human primate, a dog, cat, horse, cow, sheep, pig, or goat. The term “carrier” denotes an organic or inorganic ingredient, natural or synthetic, with which the active ingredient is combined to facilitate the application. The carriers are capable of being comingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Formulations may include a chelating agent, a buffering agent, an anti-oxidant and, optionally, an isotonicity agent, preferably pH adjusted, and a permeation/penetration enhancer. Examples of such formulations that are stable to autoclaving and long term storage are described in co-pending U.S. application Ser. No. 10/821,811, entitled “Pharmaceutical Formulation.”

Chelating agents include, for example, ethylenediaminetetraacetic acid (EDTA) and derivatives thereof, citric acid and derivatives thereof, niacinamide and derivatives thereof, sodium desoxycholate and derivatives thereof, and L-glutamic acid, N,N-diacetic acid and derivatives thereof. EDTA derivatives include dipotassium edetate, disodium edetate, calcium disodium edetate, sodium edetate, trisodium edetate, and potassium edetate.

Buffering agents include those selected from the group consisting of citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, or combinations thereof.

Antioxidants include those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate acid, sodium formaldehyde sulfoxylate, tocopheral and derivatives thereof, monothioglycerol, and sodium sulfite. The preferred antioxidant is monothioglycerol.

Isotonicity agents include those selected from the group consisting of sodium chloride, mannitol, lactose, dextrose, glycerol, and sorbitol.

Preservatives that can be used with the present compositions include benzyl alcohol, parabens, thimerosal, chlorobutanol and preferably benzalkonium chloride. Typically, the preservative will be present in a composition in a concentration of up to about 2% by weight. The exact concentration of the preservative, however, will vary depending upon the intended use and can be easily ascertained by one skilled in the art.

The compounds of the invention can be prepared in lyophilized compositions, preferably in the presence of a cryoprotecting agent such as mannitol, or lactose, sucrose, polyethylene glycol, and polyvinyl pyrrolidines. Cryoprotecting agents which result in a reconstitution pH of 6.0 or less are preferred. The invention therefore provides a lyophilized preparation of therapeutic agent(s) of the invention. The preparation can contain a cryoprotecting agent, such as mannitol or lactose, which is preferably neutral or acidic in water.

Oral, parenteral and suppository formulations of agents are well known and commercially available. The therapeutic agent(s) of the invention can be added to such well known formulations. It can be mixed together in solution or semi-solid solution in such formulations, can be provided in a suspension within such formulations or could be contained in particles within such formulations.

A product containing therapeutic agent(s) of the invention and, optionally, one or more other active agents can be configured as an oral dosage. The oral dosage may be a liquid, a semisolid or a solid. An opioid may optionally be included in the oral dosage. The oral dosage may be configured to release the therapeutic agent(s) of the invention before, after or simultaneously with the other agent (and/or the opioid). The oral dosage may be configured to have the therapeutic agent(s) of the invention and the other agents release completely in the stomach, release partially in the stomach and partially in the intestine, in the intestine, in the colon, partially in the stomach, or wholly in the colon. The oral dosage also may be configured whereby the release of the therapeutic agent(s) of the invention is confined to the stomach or intestine while the release of the other active agent is not so confined or is confined differently from the therapeutic agent(s) of the invention. For example, the therapeutic agent(s) of the invention may be an enterically coated core or pellets contained within a pill or capsule that releases the other agent first and releases the therapeutic agent(s) of the invention only after the therapeutic agent(s) of the invention passes through the stomach and into the intestine. The therapeutic agent(s) of the invention also can be in a sustained release material, whereby the therapeutic agent(s) of the invention is released throughout the gastrointestinal tract and the other agent is released on the same or a different schedule. The same objective for therapeutic agent(s) of the invention release can be achieved with immediate release of therapeutic agent(s) of the invention combined with enteric coated therapeutic agent(s) of the invention. In these instances, the other agent could be released immediately in the stomach, throughout the gastrointestinal tract or only in the intestine

The materials useful for achieving these different release profiles are well known to those of ordinary skill in the art. Immediate release is obtainable by conventional tablets with binders which dissolve in the stomach. Coatings which dissolve at the pH of the stomach or which dissolve at elevated temperatures will achieve the same purpose. Release only in the intestine is achieved using conventional enteric coatings such as pH sensitive coatings which dissolve in the pH environment of the intestine (but not the stomach) or coatings which dissolve over time. Release throughout the gastrointestinal tract is achieved by using sustained-release materials and/or combinations of the immediate release systems and sustained and/or delayed intentional release systems (e.g., pellets which dissolve at different pHs).

To improve oral bioavailability of the compounds of the present invention, excipients may be used that increase intestinal membrane permeability (Aungst, B. J. (2000) J. Pharm. Sci., vol. 89, issue 5, pp. 429-442). Permeation enhancers may include surfactants, fatty acids, medium chain glycerides, steroidal detergents, acyl carnitine and alkanoylcholines, N-acetylated alpha amino acids and N-acetylated non-alpha amino acids, and chitosans, and other mucoadhesive polymers. Specific examples include: cholate, glycocholate, glycosursodeoxycholate, ethylenediamine tetraacetic acid, hydroxypropyl-beta-cyclodextrin, hydroxypropyl-gamma-cyclodextrin, gamma-cyclodextrin, tetradecyl-beta-D-maltose, octylglucoside, citric acid, glycyrrhetinic acid, and Tween-80 (Shah, R. B. et al., J. Pharm Sci., 93(4):1070-82,2004).

In the event that it is desirable to release the therapeutic agent(s) of the invention first, the therapeutic agent(s) of the invention could be coated on the surface of the controlled release formulation in any pharmaceutically acceptable carrier suitable for such coatings and for permitting the release of the therapeutic agent(s) of the invention, such as in a temperature sensitive pharmaceutically acceptable carrier used for controlled release routinely. Other coatings which dissolve when placed in the body are well known to those of ordinary skill in the art.

The therapeutic agent(s) of the invention also may be mixed throughout a controlled release formulation, whereby it is released before, after or simultaneously with another agent. The therapeutic agent(s) of the invention may be free, that is, solubilized within the material of the formulation. The therapeutic agent(s) of the invention also may be in the form of vesicles, such as wax coated micropellets dispersed throughout the material of the formulation. The coated pellets can be fashioned to immediately release the therapeutic agent(s) of the invention based on temperature, pH or the like. The pellets also can be configured so as to delay the release of the therapeutic agent(s) of the invention, allowing the other agent a period of time to act before the therapeutic agent(s) of the invention exerts its effects. The therapeutic agent(s) of the invention pellets also can be configured to release the therapeutic agent(s) of the invention in virtually any sustained release pattern, including patterns exhibiting first order release kinetics or sigmoidal order release kinetics using materials of the prior art and well known to those of ordinary skill in the art.

The therapeutic agent(s) of the invention also can be contained within a core within the controlled release formulation. The core may have any one or any combination of the properties described above in connection with the pellets. The therapeutic agent(s) of the invention may be, for example, in a core coated with a material, dispersed throughout a material, coated onto a material or adsorbed into or throughout a material.

It should be understood that the pellets or core may be of virtually any type. They may be drug coated with a release material, drug interspersed throughout material, drug adsorbed into a material, and so on. The material may be erodible or nonerodible.

The therapeutic agent(s) of the invention, may be provided in particles. Particles as used herein means nano or microparticles (or in some instances larger) which can consist in whole or in part of the therapeutic agent(s) of the inventions or the other agents as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” These formulations may be for any mode of administration.

Delivery systems specific for the gastrointestinal tract are roughly divided into three types: the first is a delayed release system designed to release a drug in response to, for example, a change in pH; the second is a timed-release system designed to release a drug after a predetermined time; and the third is a microflora enzyme system making use of the abundant enterobacteria in the lower part of the gastrointestinal tract (e.g., in a colonic site-directed release formulation).

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestinal tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale Co., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran, et al., Science, Vol. 233: 1081 (1986)) and the group of Utah University (J. Kopecek, et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically, although not necessarily, a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the trade name EUDRAGIT); vinyl polymers and copolymers such as polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the trade name EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT I., particularly L-30D and EUDRAGIT L 100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics. In certain embodiments of the invention, the preferred enteric coating is ACRYL-EZE™ (methacrylic acid co-polymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allows optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6th Ed. (Media, PA: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. The rate of water influx and drug efflux can be controlled by the composition and size of the orifice of the device. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose dicylate, cellulose acetate succinate, cellulose propionate succinate, cellulose acetate octanoate, cellulose valerate palmitate, cellulose acetate heptanate, cellulose acetaldehyde dimethyl acetal, cellulose acetate ethylcarbamate, cellulose acetate methylcarbamate, cellulose dimethylaminoacetate and ethylcellulose.

In another embodiment, dig dosage forms are provided that comprise a sustained release coated device housing a formulation of the invention. In this embodiment, the drug-containing formulation is encapsulated in a sustained release membrane or film. The membrane may be semipermeable, as described above. A semipermeable membrane allows for the passage of water inside the coated device to dissolve the drug. The dissolved drug solution diffuses out through the semipermeable membrane. The rate of drug release depends upon the thickness of the coated film and the release of drug can begin in any part of the GI tract. Suitable membrane materials for such a membrane include ethylcellulose.

In another embodiment, drug dosage forms are provided that comprise a sustained release device housing a formulation of the invention. In this embodiment, the drug-containing formulation is uniformly mixed with a sustained release polymer. These sustained release polymers are high molecular weight water-soluble polymers, which when in contact with water, swell and create channels for water to diffuse inside and dissolve the drug. As the polymers swell and dissolve in water, more of drug is exposed to water for dissolution. Such a system is generally referred to as sustained release matrix. Suitable materials for such a device include hydropropyl methylcellulose, hydroxypropyl cellulose, hydroxyethyl cellulose and methyl cellulose.

In another embodiment, drug dosage forms are provided that comprise an enteric coated device housing a sustained release formulation of the invention. In this embodiment, the drug containing product described above is coated with an enteric polymer. Such a device would not release any drug in the stomach and when the device reaches the intestine, the enteric polymer is first dissolved and only then would the drug release begin. The drug release would take place in a sustained release fashion.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation of the compounds of the invention as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, those used for enterically coated capsules containing liquid or semisolid drug formulations.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include Endragit L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, sold under the trademark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

A product containing therapeutic agent(s) of the invention can be configured as a suppository. The therapeutic agent(s) of the invention can be placed anywhere within or on the suppository to favorably affect the relative release of the therapeutic agent(s). The nature of the release can be zero order, first order, or sigmoidal, as desired.

Suppositories are solid dosage forms of medicine intended for administration via the rectum. Suppositories are compounded so as to melt, soften, or dissolve in the body cavity (around 98.6° F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad et al., and U.S. Pat. No. 4,765,978 to Abidi et al.).

The base used in the pharmaceutical suppository composition of this invention includes, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface-active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition, may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions may be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

One or more therapeutic agents may be incorporated into the nasal delivery system or any other delivery system described herein.

A composition formulated for topical administration may be liquid or semi-solid (including, for example, a gel, lotion, emulsion, cream, ointment, spray or aerosol) or may be provided in combination with a “finite” carrier, for example, a non-spreading material that retains its form, including, for example, a patch, bioadhesive, dressing or bandage. It may be aqueous or non-aqueous; it may be formulated as a solution, emulsion, dispersion, a suspension or any other mixture.

Important modes of administration include topical application to the skin, eyes or mucosa. Thus, typical vehicles are those suitable for pharmaceutical or cosmetic application to body surfaces. The compositions provided herein may be applied topically or locally to various areas in the body of a patient. As noted above, topical application is intended to refer to application to the tissue of an accessible body surface, such as, for example, the skin (the outer integument or covering) and the mucosa (the mucous-producing, secreting and/or containing surfaces). Exemplary mucosal surfaces include the mucosal surfaces of the eyes, mouth (such as the lips, tongue, gums, cheeks, sublingual and roof of the mouth), larynx, esophagus, bronchial, nasal passages, vagina and rectum/anus; in some embodiments, preferably the mouth, larynx, esophagus, vagina and rectum/anus; in other embodiments, preferably the eyes, larynx, esophagus, bronchial, nasal passages, and vagina and rectum/anus. As noted above, local application herein refers to application to a discrete internal area of the body, such as, for example, a joint, soft tissue area (such as muscle, tendon, ligaments, intraocular or other fleshy internal areas), or other internal area of the body. Thus, as used herein, local application refers to applications to discrete areas of the body.

With respect to topical and/or local administration of the present compositions, desirable efficacy may involve, for example, penetration of therapeutic agent(s) of the invention into the skin and/or tissue to substantially reach a hyperalgesic site to provide desirable anti-hyperalgesic pain relief. The efficacy of the present compositions may be about the same as that achieved, for example, with central opiate analgesics. But, as discussed in detail herein, the efficacy achieved with therapeutic agent(s) of the invention is preferably obtained without the undesirable effects that are typically associated with central opiates including, for example, respiratory depression, sedation, and addiction, as it is believed that therapeutic agent(s) of the invention does not cross the blood brain barrier.

Also in certain embodiments, including embodiments that involve aqueous vehicles, the compositions may also contain a glycol, that is, a compound containing two or more hydroxy groups. A glycol which may be particularly useful for use in the compositions is propylene glycol. The glycol may be included in the compositions in a concentration of from greater than 0 to about 5 wt. %, based on the total weight of the composition.

For local internal administration, such as intra-articular administration, the compositions are preferably formulated as a solution or a suspension in an aqueous-based medium, such as isotonically buffered saline or are combined with a biocompatible support or bioadhesive intended for internal administration.

Lotions, which, for example, may be in the form of a suspension, dispersion or emulsion, contain an effective concentration of one or more of the compounds. The effective concentration is preferably to deliver an effective amount. For example, the compound of the present invention may find use at a concentration of between about 0.1-50% [by weight] or more of one or more of the compounds provided herein. The lotions may contain, for example, [by weight] from 1% to 50% of an emollient and the balance water, a suitable buffer, and other agents as described above. Any emollients known to those of skill in the art as suitable for application to human skin may be used. These include, but are not limited to, the following: (a) Hydrocarbon oils and waxes, including mineral oil, petrolatum, paraffin, ceresin, ozokerite, microcrystalline wax, polyethylene, and perhydrosqualene. b) Silicone oils, including dimethylpolysiloxanes, methylphenylpolysiloxanes, water-soluble and alcohol-soluble silicone-glycol copolymers. (c) Triglyceride fats and oils, including those derived from vegetable, animal and marine sources. Examples include, but are not limited to, castor oil, safflower oil, cotton seed oil, corn oil, olive oil, cod liver oil, almond oil, avocado oil, palm oil, sesame oil, and soybean oil. (d) Acetoglyceride esters, such as acetylated monoglycerides. (e) Ethoxylated glycerides, such as ethoxylated glyceryl monostearate. (f) Alkyl esters of fatty acids having 10 to 20 carbon atoms. Methyl, isopropyl and butyl esters of fatty acids are useful herein. Examples include, but are not limited to, hexyl laurate, isohexyl laurate, isohexyl palmitate, isopropyl palmitate, isopropyl myristate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. (g) Alkenyl esters of fatty acids having 10 to 20 carbon atoms. Examples thereof include, but are not limited to, oleyl myristate, oleyl stearate, and oleyl oleate. (h) Fatty acids having 9 to 22 carbon atoms. Suitable examples include, but are not limited to, pelargonic, lauric, myristic, palmitic, stearic, isostearic, hydroxystearic, oleic, linoleic, ricinoleic, arachidonic, behenic, and erucic acids. (i) Fatty alcohols having 10 to 22 carbon atoms, such as, but not limited to, lauryl, myristyl, cetyl, hexadecyl, stearyl, isostearyl, hydroxystearyl, oleyl, ricinoleyl, behenyl, erucyl, and 2-octyl dodecyl alcohols. (j) Fatty alcohol ethers, including, but not limited to ethoxylated fatty alcohols of 10 to 20 carbon atoms, such as, but are not limited to, the lauryl, cetyl, stearyl, isostearyl, oleyl, and cholesterol alcohols having attached thereto from 1 to 50 ethylene oxide groups or 1 to 50 propylene oxide groups or mixtures thereof. (k) Ether-esters, such as fatty acid esters of ethoxylated fatty alcohols (l) Lanolin and derivatives, including, but not limited to, lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases. (m) polyhydric alcohols and polyether derivatives, including, but not limited to, propylene glycol, dipropylene glycol, polypropylene glycol [M.W. 2000-4000], polyoxyethylene polyoxypropylene glycols, polyoxypropylene polyoxyethylene glycols, glycerol, ethoxylated glycerol, propoxylated glycerol, sorbitol, ethoxylated sorbitol, hydroxypropyl sorbitol, polyethylene glycol [M.W. 200-6000], methoxy polyethylene glycols 350, 550, 750, 2000, 5000, poly(ethylene oxide) homopolymers [M.W. 100,000-5,000,000], polyalkylene glycols and derivatives, hexylene glycol (2-methyl-2,4-pentanediol), 1,3-butylene glycol, 1,2,6,-hexanetriol, ethohexadiol USP (2-ethyl-1,3-hexanediol), C₁₅-C₁₈ vicinal glycol and polyoxypropylene derivatives of trimethylolpropane. (n) polyhydric alcohol esters, including, but not limited to, ethylene glycol mono- and di-fatty acid esters, diethylene glycol mono- and di-fatty acid esters, polyethylene glycol [M.W. 200-6000], mono- and di-fatty esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. (o) Wax esters, including, but not limited to, beeswax, spermaceti, myristyl myristate, and stearyl stearate and beeswax derivatives, including, but not limited to, polyoxyethylene sorbitol beeswax, which are reaction products of beeswax with ethoxylated sorbitol of varying ethylene oxide content that form a mixture of ether-esters. (p) Vegetable waxes, including, but not limited to, carnauba and candelilla waxes. (q) phospholipids, such as lecithin and derivatives. (r) Sterols, including, but not limited to, cholesterol and cholesterol fatty acid esters. (s) Amides, such as fatty acid amides, ethoxylated fatty acid amides, and solid fatty acid alkanolamides.

The lotions further preferably contain [by weight] from 1% to 10%, more preferably from 2% to 5%, of an emulsifier. The emulsifiers can be nonionic, anionic or cationic. Examples of satisfactory nonionic emulsifiers include, but are not limited to, fatty alcohols having 10 to 20 carbon atoms, fatty alcohols having 10 to 20 carbon atoms condensed with 2 to 20 moles of ethylene oxide or propylene oxide, alkyl phenols with 6 to 12 carbon atoms in the alkyl chain condensed with 2 to 20 moles of ethylene oxide, mono- and di-fatty acid esters of ethylene oxide, mono- and di-fatty acid esters of ethylene glycol where the fatty acid moiety contains from 10 to 20 carbon atoms, diethylene glycol, polyethylene glycols of molecular weight 200 to 6000, propylene glycols of molecular weight 200 to 3000, glycerol, sorbitol, sorbitan, polyoxyethylene sorbitol, polyoxyethylene sorbitan and hydrophilic wax esters. Suitable anionic emulsifiers include, but are not limited to, the fatty acid soaps, e.g., sodium, potassium and triethanolamine soaps, where the fatty acid moiety contains from 10 to 20 carbon atoms. Other suitable anionic emulsifiers include, but are not limited to, the alkali metal, ammonium or substituted ammonium alkyl sulfates, alkyl arylsulfonates, and alkyl ethoxy ether sulfonates having 10 to 30 carbon atoms in the alkyl moiety. The alkyl ethoxy ether sulfonates contain from 1 to 50 ethylene oxide units. Among satisfactory cationic emulsifiers are quaternary ammonium, morpholinium and pyridinium compounds. Certain of the emollients described in preceding paragraphs also have emulsifying properties. When a lotion is formulated containing such an emollient, an additional emulsifier is not needed, though it can be included in the composition.

The balance of the lotion is water or a C₂ or C₃ alcohol, or a mixture of water and the alcohol. The lotions are formulated by simply admixing all of the components together. Preferably the compound, such as loperamide, is dissolved, suspended or otherwise uniformly dispersed in the mixture.

Other conventional components of such lotions may be included. One such additive is a thickening agent at a level from 1% to 10% by weight of the composition. Examples of suitable thickening agents include, but are not limited to: cross-linked carboxypolymethylene polymers, ethyl cellulose, polyethylene glycols, gum tragacanth, gum kharaya, xanthan gums and bentonite, hydroxyethyl cellulose, and hydroxypropyl cellulose.

Creams can be formulated to contain a concentration effective to deliver an effective amount of therapeutic agent(s) of the invention to the treated tissue, typically at between about 0.1%, preferably at greater than 1% up to and greater than 50%, preferably between about 3% and 50%, more preferably between about 5% and 15% therapeutic agent(s) of the invention. The creams also contain from 5% to 50%, preferably from 10% to 25%, of an emollient and the remainder is water or other suitable non-toxic carrier, such as an isotonic buffer. The emollients, as described above for the lotions, can also be used in the cream compositions. The cream may also contain a suitable emulsifier, as described above. The emulsifier is included in the composition at a level from 3% to 50%, preferably from 5% to 20%.

These compositions that are formulated as solutions or suspensions may be applied to the skin, or, may be formulated as an aerosol or foam and applied to the skin as a spray-on. The aerosol compositions typically contain [by weight] from 25% to 80%, preferably from 30% to 50%, of a suitable propellant. Examples of such propellants are the chlorinated, fluorinated and chlorofluorinated lower molecular weight hydrocarbons. Nitrous oxide, carbon dioxide, butane, and propane are also used as propellant gases. These propellants are used as understood in the art in a quantity and under a pressure suitable to expel the contents of the container.

Suitably prepared solutions and suspensions may also be topically applied to the eyes and mucosa. Solutions, particularly those intended for ophthalmic use, may be formulated as 0.01%-10% isotonic solutions, pH about 5-7, with appropriate salts, and preferably containing one or more of the compounds herein at a concentration of about 0.1%, preferably greater than 1%, up to 50% or more. Suitable ophthalmic solutions are known [see, e.g., U.S. Pat. No. 5,116,868, which describes typical compositions of ophthalmic irrigation solutions and solutions for topical application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D.L.-sodium, .β.-hydroxybutyrate and 5-5.5 mM glucose.

Gel compositions can be formulated by simply admixing a suitable thickening agent to the previously described solution or suspension compositions. Examples of suitable thickening agents have been previously described with respect to the lotions.

The gelled compositions contain an effective amount of therapeutic agent(s) of the invention, typically at a concentration of between about 0.1-50% by weight or more of one or more of the compounds provided herein; from 5% to 75%, preferably from 10% to 50%, of an organic solvent as previously described; from 0.5% to 20%, preferably from 1% to 10% of the thickening agent; the balance being water or other aqueous or non-aqueous carrier, such as, for example, an organic liquid, or a mixture of carriers.

The formulations can be constructed and arranged to create steady state plasma levels. Steady state plasma concentrations can be measured using HPLC techniques, as are known to those of skill in the art. Steady state is achieved when the rate of drug availability is equal to the rate of drug elimination from the circulation. In typical therapeutic settings, the therapeutic agent(s) of the invention will be administered to patients either on a periodic dosing regimen or with a constant infusion regimen. The concentration of drug in the plasma will tend to rise immediately after the onset of administration and will tend to fall over time as the drug is eliminated from the circulation by means of distribution into cells and tissues, by metabolism, or by excretion. Steady state will be obtained when the mean drug concentration remains constant over time. In the case of intermittent dosing, the pattern of the drug concentration cycle is repeated identically in each interval between doses with the mean concentration remaining constant. In the case of constant infusion, the mean drug concentration will remain constant with very little oscillation. The achievement of steady state is determined by means of measuring the concentration of drug in plasma over at least one cycle of dosing such that one can verify that the cycle is being repeated identically from dose to dose. Typically, in an intermittent dosing regimen, maintenance of steady state can be verified by determining drug concentrations at the consecutive troughs of a cycle, just prior to administration of another dose. In a constant infusion regimen where oscillation in the concentration is low, steady state can be verified by any two consecutive measurements of drug concentration.

Included within embodiments, is a kit which includes a container containing an opioid formulation and a container containing a compound of the present disclosure. The kit may include a pharmaceutical preparation vial, and a pharmaceutical preparation diluents vial. The diluents vial may, for example, contain diluents such as physiological saline for diluting what could be a concentrated solution or lyophilized powder of the compound. The instructions can include instructions for mixing a particular amount of the diluents with a particular amount of the concentrated pharmaceutical preparation, whereby a final formulation for injection or infusion is prepared. The instructions may include instructions for treating a patient with an effective amount of the compound. It also will be understood that the containers containing the preparations, whether the container is a bottle, a vial with a septum, an ampoule with a septum, an infusion bag, and the like, can contain additional indicia such as conventional markings which change color when the preparation has been autoclaved or otherwise sterilized.

### EXAMPLE 1

### 17-allyl-17-cyclopropylmethyl-4,5α-epoxy-3,14-dihydroxy-6-oxomorphinanium iodide (D0001)

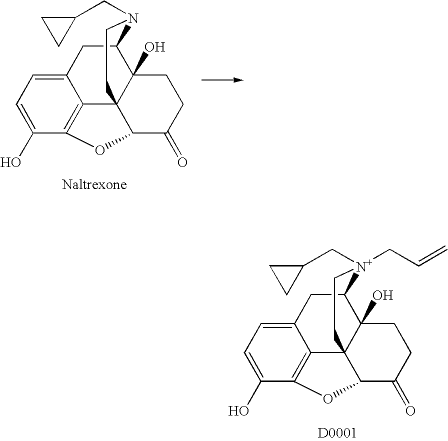

Naltrexone (2.0 g, 5.86 mmol) was dissolved in DMF (10 mL, anhydrous) under nitrogen. Allyl iodide (0.5 ml., 5.18 mmol) was added. The mixture was stirred at room temperature for 4 days. DMF was removed. The residue was stirred with 50 mL of water for 10 min. The aqueous solution was separated from the solid precipitates and washed with dichloromethane (50 mL). It was lyophilized to give a hygroscopic solid (1.2 g). 0.2 Gram of this solid was dissolved in water (30 mL). The pH of the water solution was adjusted to 10 by Na₂CO₃. This solution was washed with dichloromethane (2×20 mL) and lyophilized to give a yellow solid. This solid was purified by a reverse phase column (4 g, C-18) to 28 mg of a solid containing product D0001.

### EXAMPLE 2

### 17-Isobutyl-4,5α-epoxy-3,14-dihydroxy-17-methyl-6-oxomorphinanium triflate (D0002)

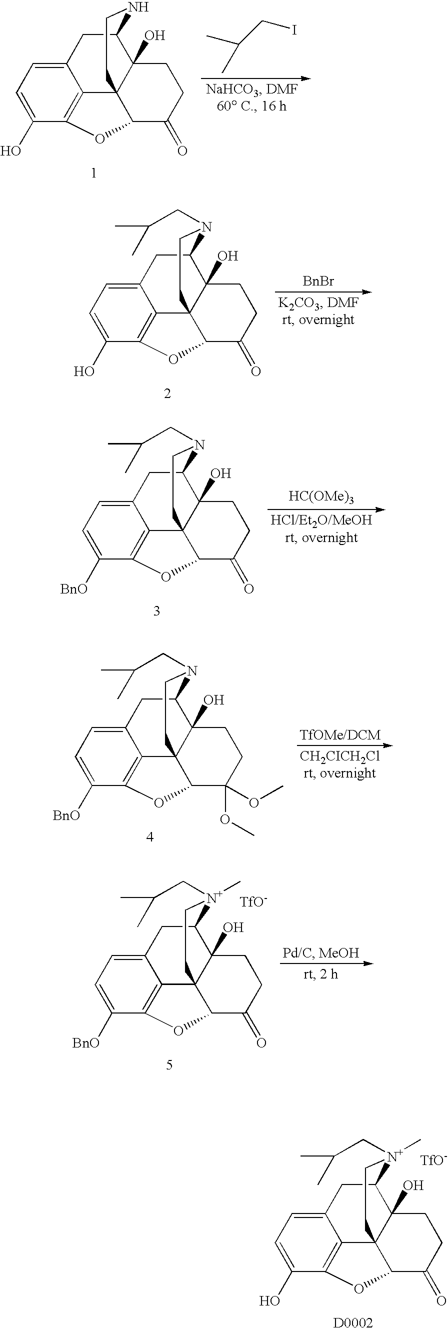

### (i) 17-Isobutyl-4,5α-epoxy-3,14-dihydroxymorphinan-6-one (2)

A mixture of noroxymorphone 1 (0.574 g, 2 mmol), isobutyl iodide (0.253 ml, 2.2 mmole) and NaHCO₃ (0.184 g, 2.2 mmole) in DMF (6 ml) was heated to 90° C. for 8 h under N₂. The solvent was evaporated to dryness and purified by column chromatography using 4% NH₄OH+4% MeOH+ethyl acetate as eluent to get 0.505 g (67%) of the product 2. ¹H NMR showed complex spectra, so identified by mass spectrum {(APCI⁺): 344 (M+1)} and carried to the next step.

### (ii) 17-Isobutyl-4,5α-epoxy-3-benzyloxy-14-hydroxymorphinan-6-one (3)

Compound 2 (560 mg, 1.63 mmol) and K₂CO₃ (562 mg, 4.08 mmol) were combined in anhydrous DMF (20 mL). Benzyl bromide (0.21 mL, 1.80 mmol) was added. The resulting mixture was stirred at room temperature under N₂ overnight. Mass spectrometry showed complete consumption of 1. EtOAc (100 mL) was added. The solution was washed with water (3×60 mL) and brine (60 mL), dried over Na₂SO₄ and filtered. The filtrate was evaporated. The yellow gummy solid residue was purified by column (eluent: 5-50% EtOAc in hexanes) to give 3 (510 mg, 72%) as a white solid. ¹H NMR (300 MHz, CDCl₃) δ ppm 7.43-7.50 (m, 2H), 7.29-7.41 (m, 3H), 6.72 (d, J=8.0 Hz, 1H), 6.58 (d, J=8.3 Hz, 1H), 5.22 (s, 1H). 5.18-5.34 (m, 2H), 4.70 (s, 1H), 2.88-3.17 (m, 3H), 2.09-2.66 (m, 7H), 1.59-1.95 (m, 4H), 0.95 (t, J=6.6 Hz, 6H). MS [M+H]: 434.2.

### (iii) 17-Isobutyl-4,5α-epoxy-3-benzyloxy-14-hydroxymorphinan-6-one dimethyl ketal (4)

Compound 3 (134 mg, 0.31 mmol) and HC(OMe)₃ (0.34 mL, 3.1 mmol) were combined in anhydrous MeOH (10 mL). HCl (0.15 mL, 4 M in dioxane, 0.6 mmol) was added. The resulting mixture was stirred at room temperature under N₂ overnight. Mass spectrometry showed complete consumption of 3. Na₂CO₃ (2 M, 10 mL) was added. MeOH was removed, and the aqueous solution was extracted with DCM (3×20 mL). The DCM extracts were combined, dried over Na₂SO₄ and filtered. The filtrate was evaporated. The yellow gummy solid product 4 (126 mg, 85%) was used in the next reaction without purification. MS [M+H]: 480.3.

### (iv) 17-Isobutyl-4,5α-epoxy-3-benzyloxy-14-hydroxy-17-methyl-6 oxo-morphinanium triflate (5)

Compound 4 (126 mg, 0.263 mmol, crude product from the above reaction) was dissolved in anhydrous 1,2-dichloroethane (5 mL). Methyl triflate (0.78 M in DCM, 1.68 mL, 1.31 mmol) was added. The resulting mixture was stirred at room temperature under N₂ for 2d. Mass spectrometry showed >60% conversion. The reaction solution was applied to a column of 20 g of silica gel and purified (eluent: 5-10% MeOH in DCM) to give 5 (91.6 mg, 60%) as a white solid. ¹H NMR (300 MHz, METHANOL-d₃) δ ppm 7.26-7.48 (m, 5H), 6.90 (d, J=8.3 Hz, 1H), 6.80 (d, J=8.3 Hz, 1H), 5.25 (d, J=2.8 Hz, 2H), 4.94 (s, 1H), 3.95 (d, J=5.0 Hz, 1H), 3.71 (s, 3H), 3.60-3.69 (m, 1H), 3.34-3.55 (m, 3H), 2.91-3.24 (m, 5H), 2.34-2.47 (m, 1H), 2.18-2.29 (m, 1H), 2.00-2.10 (m, 1H), 1.64-1.82 (m, 2H), 1.22 (dd, J=12.4, 6.6 Hz, 6H). MS [M+H]: 448.3.

### (iv) 17-Isobutyl-4,5α-epoxy-3,14-dihydroxy-17-methyl-6-oxo morphinanium triflate (D0002)

Compound 5 (91.6 mg, 0.158 mmol) was dissolved in MeOH (10 mL). Pd/C (92 mg, 10%, wet, 0.086 mmol) was added. The resulting mixture was stirred at room temperature under a H₂ balloon. Mass spectrometry after 35 min indicated complete conversion of the starting material to the product. The reaction solution was filtered through a pad of Celite. The Celite was washed with MeOH (2×10 mL). The filtrate was evaporated. The residue was dissolved in water (3 mL) and lyophilized to give D0002 (71.6 mg, 92%) as a white solid. ¹H NMR (300 MHz, D₂O) δ ppm 6.77 (d, J=8.3 Hz, 1H), 6.75 (d, J=8.5 Hz, 1H), 4.98 (s, 1H), 3.98 (d, J=5.0 Hz, 1H), 3.61 (s, 3H), 3.63 (d, J=4.1 Hz, 1H), 3.27-3.54 (m, 2H), 2.73-3.21 (m, 5H), 2.16-2.39 (m, 2H), 1.97-2.09 (m, 1H), 1.68-1.83 (m, 2H), 1.10 (dd, J=12.4, 6.6 Hz, 6H). HPLC purity: 100%. MS [M+H]: 358.1.

### EXAMPLE 3

### 17-(3,3′-dimethylallyl)-4,5α-epoxy-3,14-dihydroxy-17-methyl-6-oxomorphinanium iodide (D0003)

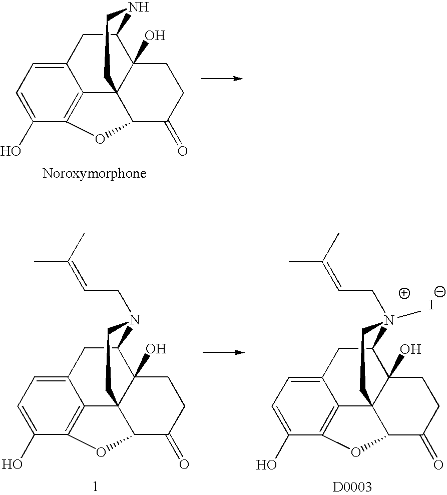

### (i) 17-(3,3′-dimethylallyl)-4,5α-epoxy-3,14-dihydroxy-17-methyl-6-oxo morphinanium bromide (1)

To a solution of noroxymorphone (498 mg, 1 eq.) in 5 mL of DMF was added sodium bicarbonate (160 mg, 1.1 eq.) and allylbromide (222 μL, 1.1 eq.). The reaction mixture was stirred overnight at 90° C. The reaction mixture was cooled down to room temperature and diluted with chloroform (20 mL) and washed with brine. The aqueous washings were extracted (3×50 mL) with chloroform and the organics were pooled. The combined chloroform extracts were dried over anhydrous Mg₂SO₄ and concentrated. The crude product 1 was purified by silica column chromatography (10 g SiO₂) using dichloromethane-methanol (98:2) as eluent to afford 388 mg (63%) of the intermediate compound 1.

### (ii) 17-(3,3′-dimethylallyl)-4,5α-epoxy-3,14-dihydroxy-17-methyl-6-oxo morphinanium iodide

To a solution of compound 1 (388 mg, 1 eq.) in 2 mL of DMF was added methyl iodide (680 μL, 10 eq.) and stirred at 55° C. for 5 days. A sample was analyzed by HPLC to determine the percentage of conversion. The crude reaction mixture was partitioned between dichloromethane and sodium bicarbonate solution (pH>10). The aqueous phase was lyophilized to get a solid which was purified by passing through a reverse phase C-18 column using water-methanol solvent mixture as eluent (gradient elution) to afford a white solid which was again purified by semi-prep HPLC to afford the product D0003.

### EXAMPLES 4 AND 5

### 17-Cyclopropylmethyl-4,5α-epoxy-3,14-dihydroxy-17-methyl-6α-methoxy morphinanium trifluoroacetate (D0004) and 17-Cyclopropylmethyl-4,5α-epoxy-3,14-dihydroxy-17-methyl-60-methoxy morphinanium trifluoroacetate (D0005)

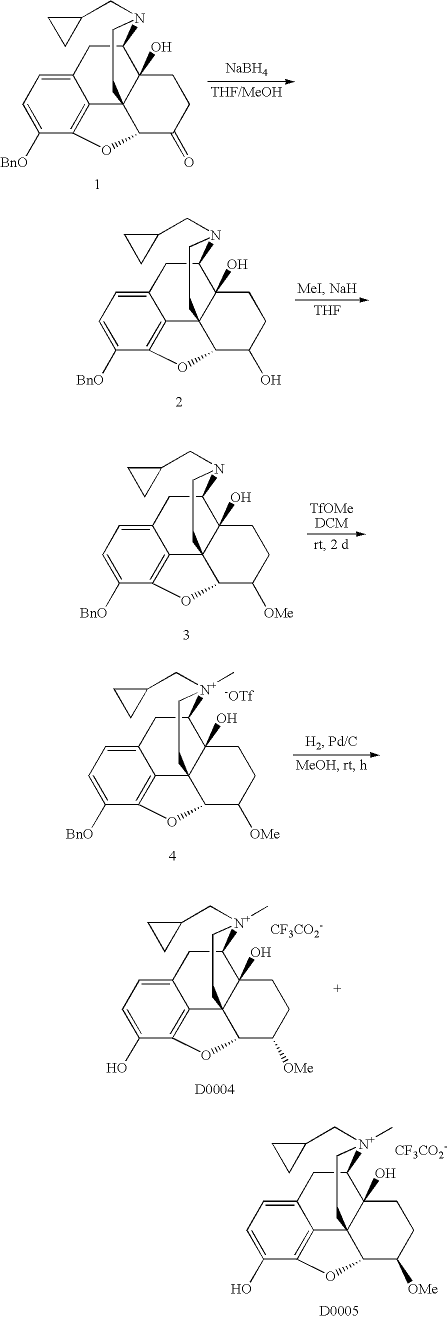

### (i) 17-Cyclopropylmethyl-4,5α-epoxy-3-benzyloxy-6,14-dihydroxymorphinan (2)

Compound 1 (1.64 g, 3.8 mmol), prepared by the method for compounds D0010-D0013, below, was dissolved in a mixture of THF (35 mL) and MeOH (35 mL) and stirred at 0° C. NaBH₄ (0.29 g, 7.63 mmol) was added. The resulting reaction solution was stirred for 1 h Water (110 mL) was added and the mixture was extracted with DCM (3×100 mL). The DCM extracts were combined, dried over Na₂SO₄ and filtered. The filtrate was evaporated and the residue was purified by column (eluent: 3-10% MeOH in DCM) to give 2 (1.39 g, 84%) as a white foam. This compound has a complex ¹H NMR because it is a mixture of 6α- and 6β-hydroxyl isomers. MS showed the correct molecular weight: [M+H]: 434.

### (ii) 17-Cyclopropylmethyl-4,5α-epoxy-3-benzyloxy-14-hydroxy-6-methoxymorphinan (3)

Compound 2 (1.24 g, 3.8 mmol) was dissolved in anhydrous THF (50 mL) and stirred at room temperature under N₂. MeI (0.66 mL, 6.86 mmol) was added, followed by NaH (172 mg, 60% in mineral oil, 4.29 mmol). The resulting reaction solution was stirred for 17 h. The reaction was quenched with water (1.0 mL) and the volatiles were removed. The residue was purified by column (eluent: 50-100% EtOAc in hexanes) to give 3 (0.37 g, 29%) as a white foam. This compound has a complex ¹H NMR because it is a mixture of 6α- and 6β-methoxy isomers. MS showed the correct molecular weight: [M+H]: 448.

### (iii) 17-Cyclopropylmethyl-4,5α-epoxy-3-benzyloxy-14-hydroxy-17-methyl-6-methoxy morphinanium triflate (4)

Compound 3 (0.37 g, 0.83 mmol) was dissolved in anhydrous DCM (10 mL) and stirred at room temperature under N₂. TfOMe (2.4 mL, 0.69 M in DCM, 1.66 mmol) was added and the resulting solution was stirred for 22 h. Aqueous Na₂CO₃ (5 mL, 2 M) was added and the mixture was extracted with DCM (2×20 mL). The DCM extracts were combined, dried over Na₂SO₄ and filtered. The filtrate was evaporated and the residue was purified by column (eluent: 3-10% MeOH in DCM) to give 4 (154 mg, 30%) as a white foam. This compound has a complex ¹H NMR because it is a mixture of 6α- and 6β-methoxy isomers. MS showed the correct molecular weight: [M+H]: 462.

### (iv) 17-Cyclopropylmethyl-4,5α-epoxy-3,14-dihydroxy-17-methyl-6α-methoxy morphinanium trifluoroacetate (D0004) and (R)-17-Cyclopropylmethyl-4,5α-epoxy-3,14-dihydroxy-17-methyl-6β-methoxy morphinanium trifluoroacetate (D0005)

Compound 4 (150 mg, 0.25 mmol) was dissolved in MeOH (10 mL). Pd/C (152 mg, 10%, wet, 0.141 mmol) was added. The resulting mixture was stirred at room temperature under a H₂ balloon. Mass spectrometry after 2.5 h indicated complete conversion of the starting material to the product. The reaction solution was filtered. The filtrate was evaporated and the residue was purified by semi-prep HPLC to give D0004 (29 mg, 21%) as a white foam and D0005 (45 mg, 32%) as a white foam.

D-0004: ¹H NMR (300 MHz, D₂O) δ ppm 6.78 (d, J=8.3 Hz, 1H), 6.66 (d, J=8.3 Hz, 1H), 4.91 (d, J=5.2 Hz, 1H), 3.81-4.00 (m, 3H), 3.57 (s, 3H), 3.59 (d, J=21.0 Hz, 1H), 3.32 (s, 3H), 2.96-3.29 (m, 3H), 2.51-2.82 (m, 2H), 1.67-1.90 (m, 2H), 1.54-1.64 (m, 2H), 1.22-1.39 (m, 1H), 1.05-1.21 (m, 1H), 0.64-0.92 (m, 2H), 0.45-0.59 (m, 1H), 0.26-0.42 (m, 1H). HPLC purity: 100%. MS [M+H]: 372.2.

D-0005: ¹H NMR (300 MHz, D₂O) δ ppm 6.80 (d, J=8.3 Hz, 1H), 6.73 (d, J=8.3 Hz, 1H), 4.57 (d, J=6.3 Hz, 1H), 3.84-3.96 (m, 2H), 3.50-3.65 (m, 4H), 3.36 (s, 3H), 3.05-3.29 (m, 3H), 2.83-3.00 (m, 1H), 2.48-2.74 (m, 2H), 1.40-1.91 (m, 5H), 1.05-1.20 (m, 1H), 0.77-0.91 (m, 1H), 0.66-0.77 (m, 1H), 0.45-0.60 (m, 1H), 0.25-0.38 (m, 1H). HPLC purity: 100%. MS [M+H]: 372.2.

### EXAMPLE 6

### 17-[(2′-tetrahydrofuryl)methyl]-4,5α-epoxy-3,14-dihydroxy-17-methyl-morphinanium-6-one trifluoroacetate (D0006)

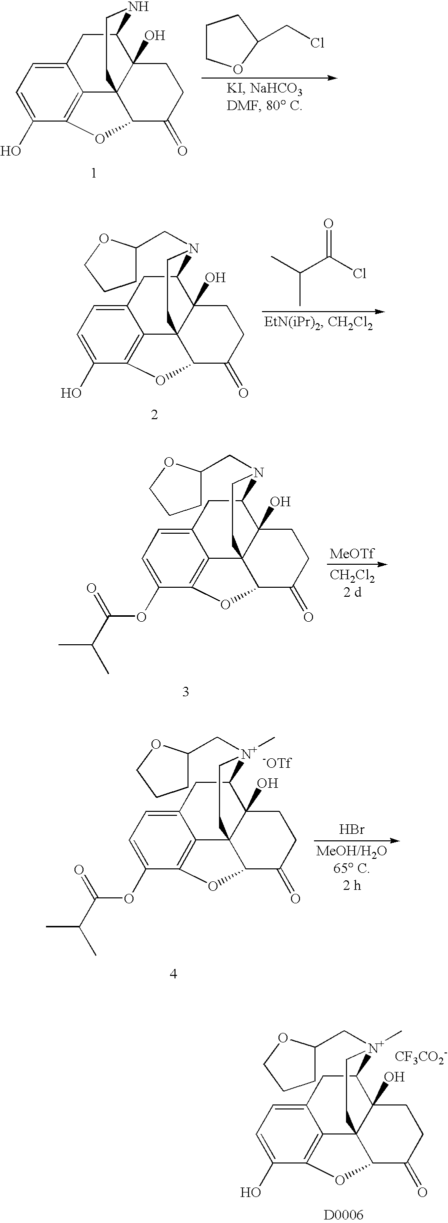

### (i) 17-[(2′-tetrahydrofuryl)methyl]-4,5α-epoxy-3,14-dihydroxymorphinan-6-one (2)

Noroxymorphone 1 (1.0 g, 3.48 mmol), 2-chloromethyltetrahydrofuran (1.51 mL, 13.9 mmol), KI (1.16 g, 7.0 mmol) and NaHCO₃ (1.16 g, 13.9 mmol) were combined in anhydrous DMF (20 mL) and stirred at 90-100° C. under N₂. Mass spectrometry after 19 h showed most of I had been consumed. The reaction solution was cooled to room temperature and water (80 mL) was added. This mixture was extracted with DCM (2×70 mL). The DCM extracts were combined, dried over Na₂SO₄ and filtered. The filtrate was evaporated. The brown gummy solid was purified by column chromatography (eluent: 0-5% MeOH in DCM) to give 2 (1.14, 88%) as a yellow gum. ¹H NMR (300 MHz, CDCl₃) δ ppm 6.72 (d, J=8.0 Hz, 1H), 6.60 (d, J=8.3 Hz, 1H), 4.67 (s, 1H), 3.61-4.34 (m, 5H), 2.99-3.18 (m, 2H), 2.51-2.78 (m, 4H), 2.22-2.50 (m, 3H), 1.73-2.14 (m, 7H), 1.46-1.71 (m, 3H). MS [M+H]: 372.2

### (ii) 17-[(2′-tetrahydrofuryl)methyl]-4,5α-epoxy-3-isobutryloxy-14-hydroxymorphinan-6-one (3)

Compound 2 (1.14 g, 3.08 mmol) was dissolved in anhydrous DCM (60 mL) and stirred at 0° C. under N₂. EtN(iPr)₂ (2.13 mL, 12.28 mmol) was added, followed by dropwise addition of isobutyryl chloride (0.65 mL, 6.15 mmol). The resulting mixture was stirred at room temperature for 4 h. Aqueous Na₂CO₃ (60 mL, 0.5 N) was added and the mixture was extracted with DCM (2×60 mL). The DCM extracts were combined, dried over Na₂SO₄ and filtered. The filtrate was evaporated. The brown gummy solid was purified by column (eluent: EtOAc) to give 7 (202 mg, 15%) as a yellow foam. ¹H NMR (300 MHz, CDCl₃) δ ppm 6.84 (d, J=8.3 Hz, 1H), 6.69 (d, J=8.0 Hz, 1H), 5.12 (s, 1H), 4.69 (s, 1H), 3.97-4.09 (m, 1H), 3.85-3.95 (m, 1H), 3.72-3.82 (m, 1H), 2.21-3.20 (m, 10H), 1.81-2.12 (m, 4H), 1.51-1.69 (m, 6H), 1.33 (d, J=7.4 Hz, 6H). MS [M+H]: 442.2.

### (iii) 17-[(2′-tetrahydrofuryl)methyl]-4,5α-epoxy-3-isobutyryloxy-14-hydroxy-17-methyl-morphinanium-6-one triflate (4)

Compound 3 (202 mg, 0.46 mmol) was dissolved in anhydrous DCM (10 mL) and stirred at room temperature under N₂. TfOMe (2.4 mL, 0.69 M in DCM, 1.66 mmol) was added and the resulting solution was stirred for 22 h. Aqueous Na₂CO₃ (5 mL, 2M) was added and the mixture was extracted with DCM (2×20 mL). The DCM extracts were combined, dried over Na₂SO₄ and filtered. The filtrate was evaporated and the residue was purified by column (eluent: 2-10% MeOH in DCM) to give 8 (113 mg, 41%) as a white foam. ¹H NMR (300 MHz, CDCl₃) δ ppm 6.96 (d, J=8.0 Hz, 1H), 6.82 (d, J=8.3 Hz, 1H), 5.66 (s, 1H), 4.78 (s, 1H), 4.64 (d, J=3.6 Hz, 1H), 4.35-4.48 (m, 1H), 3.99 (s, 3H), 3.80-3.96 (m, 2H), 2.79-3.71 (m, 10H), 2.39-2.51 (m, 1H), 2.18-2.36 (m, 2H), 1.92-2.06 (m, 2H), 1.84 (d, J=10.7 Hz, 1H), 1.52-1.73 (m, 3H), 1.34 (d, J=6.9 Hz, 6H). MS [M+H]: 456.2.

### (iv) 17-[(2′-tetrahydrofuryl)methyl]-4,5α-epoxy-3,14-dihydroxy-17-methyl-morphinanium-6-one trifluoroacetate (D-0006)

Compound 4 (112 mg, 0.185 mmol) was dissolved in MeOH (10 mL) and aqueous HBr (0.4 mL, 48%) was added. The resulting solution was stirred at 70° C. for 2.5 h. Solvents were evaporated and the residue was purified by semi-prep HPLC to give D-0006 (27 mg, 26%) as a white foam. ¹H NMR (300 MHz, D₂O) δ ppm 6.77 (d, J=8.3 Hz, 1H), 6.70 (d, J=8.3 Hz, 1H), 4.97 (s, 1H), 4.44-4.56 (m, 1H), 4.01 (d, J=3.9 Hz, 1H), 3.82 (d, J=7.2 Hz, 3H), 3.68 (s, 3H), 3.28-3.45 (m, 3H), 2.74-3.19 (m, 4H), 2.14-2.28 (m, 2H), 1.98-2.08 (m, 1H), 1.66-1.96 (m, 4H), 1.51-1.65 (m, 1H). HPLC purity: 100%. MS [M+H]: 386.2.

### EXAMPLE 7

### 4,5α-epoxy-3-hydroxy-(17,14-N,O-ethylene)morphinanium-6-one trifluoroacetate (D0007)

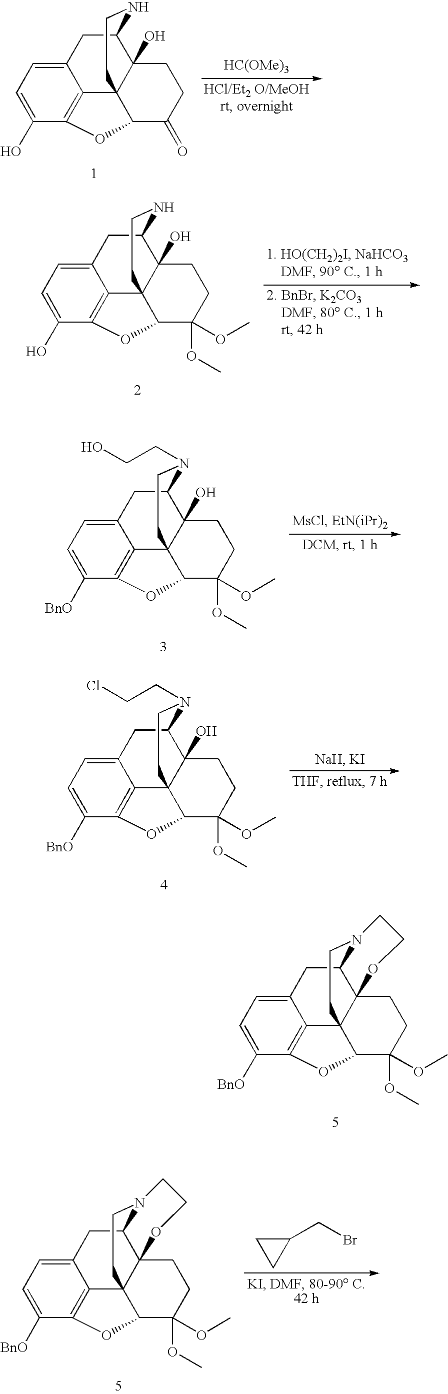

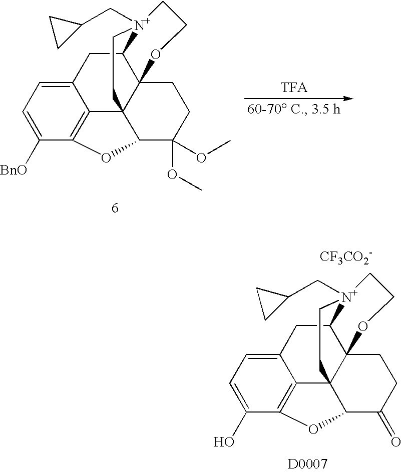

### (i) Noroxymorphone dimethyl ketal (2)

Noroxymorphone 1 (3.0 g, 10.4 mmol) was suspended in anhydrous MeOH (100 mL). Trimethyl orthoformate (3.4 mL, 31.2 mmol) was added, followed by HCl (2 M in Et₂O, 15.6 mL, 31.2 mmol). The resulting mixture was stirred at room temperature for 21 h. Na₂CO₃ (2 M, 50 mL, 100 mmol) was added. After 10 min of stirring MeOH was removed by rotary evaporation. The aqueous residue was diluted with water (50 mL) and extracted with DCM (3×60 mL). The DCM extracts were combined, dried over Na₂SO₄ and filtered. The filtrate was evaporated. The white solid product (2.6 g, 75%) was used in the next reaction without purification. MS [M+H]: 334.2.

### (ii) 3-Benzyloxy-4,5α-epoxy-17-(2′-hydroxyethyl)morphinan-6-one dimethyl ketal (3)

Noroxymorphone dimethyl ketal 2 (2.4 g, 7.2 mmol), 2-iodoethanol (0.67 mL, 8.65 mmol) and NaHCO₃ (0.79 g, 9.36 mmol) were combined in anhydrous DMF (50 mL) and heated at 80-90° C. under N₂ for 1 h. Benzyl bromide (0.97 mL, 8.65 mmol) was added, followed by K₂CO₃ (1.98 g, 14.4 mmol). The resulting mixture was stirred for 2 h and cooled to room temperature. More K₂CO₃ (3.80 g, 27.5 mmol) was added and stirring was continued for another 18 h. More benzyl bromide (0.5 mL, 4.3 mmol) was added and the reaction was continued for another 24 h. The reaction solution was diluted with EtOAc (150 mL) and washed with water (3×80 mL) and brine (80 mL). It was dried over Na₂SO₄ and filtered. The filtrate was evaporated and residue was purified by column (eluent: 30-100% EtOAc in hexanes then 10% MeOH in DCM) to give 3 (0.91 g, 27%) as a yellow gum. ¹H NMR (300 MHz, CDCl₃) δ ppm 7.28-7.49 (m, 5H), 6.75 (d, J=8.3 Hz, 1H), 6.53 (d, J=8.3 Hz, 1H), 5.16-5.34 (m, 2H), 4.59 (s, 1H), 3.63-3.72 (m, 2H), 3.38 (s, 3H), 3.27-3.46 (m, 1H), 3.17 (s, 3H), 2.86-3.11 (m, 4H), 2.52-2.78 (m, 4H), 2.28-2.42 (m, 2H), 1.84-1.98 (m, 1H), 1.36-1.68 (m, 3H). MS [M+H]: 468.2.

### (iii) 3-Benzyloxy-4,5α-epoxy-17-(2′-chloroethyl)morphinan-6-one dimethyl ketal (4)

Morphinan 3 (0.91 g, 1.95 mmol) was dissolved in anhydrous DCM (20 mL) and stirred under N₂. EtN(iPr)₂ (1.06 ml, 5.85 mmol) was added, followed by methanesulfonyl chloride (0.17 mL, 2.15 mmol). The resulting mixture was stirred at room temperature for 1 h. DCM was removed and the residue was purified by column (eluent: 20-50% EtOAc in hexanes) to give 4 (454 mg, 48%) as a white solid. ¹H NMR (300 MHz, CDCl₃) δ ppm 7.29-7.50 (m, 5H), 6.75 (d, J=8.3 Hz, 1H), 6.53 (d, J=8.5 Hz, 1H), 5.15-5.35 (m, 2H), 5.01 (s, 1H), 4.62 (s, 1H), 3.59 (br. s., 2H), 3.40 (s, 3H), 3.08 (s, 3H), 2.48-3.01 (m, 6H), 2.25-2.42 (m, 2H), 1.89-2.03 (m, 1H), 1.34-1.71 (m, 4H). MS [M+H]: 486.2

### (iv) 3-Benzyloxy-4,5α-epoxy-(17,14-N,O-ethylene)morphinan-6-one dimethyl ketal (5)

Compound 4 (452 mg, 0.932 mmol) and KI (295 mg, 1.77 mmol) were combined in anhydrous THF (50 mL) and stirred under N₂. NaH (123 mg, 60% in mineral oil, 3.08 mmol) was added and the resulting mixture was heated at reflux for 7 h. After it was cooled to room temperature water (0.5 mL) was added. Stirring was continued for 10 min and solvents were removed. The residue was purified by column (eluent: 4-10% MeOH in DCM) to give 5 (399 mg, 95%) as a white solid. ¹H NMR (300 MHz, CDCl₃) δ ppm 7.28-7.49 (m, 5H), 6.77 (d, J=8.3 Hz, 1H), 6.56 (d, J=8.3 Hz, 1H), 5.31 (s, 1H), 5.16-5.34 (m, 2H), 4.61 (s, 1H), 3.83-3.92 (m, 2H), 3.49-3.65 (m, 2H), 3.39 (s, 3H), 3.01-3.03 (m, 3H), 2.93-3.19 (m, 3H), 2.50-2.70 (m, 2H), 1.86-2.00 (m, 1H), 1.58-1.70 (m, 1H), 1.37-1.57 (m, 2H), 1.15-1.28 (m, 1H). MS [M+H]: 450.2.

### (v) 3-Benzyloxy-17-cyclopropylmethyl-4,5α-epoxy-(17,14-N,O-ethylene) morphinanium-6-one dimethyl ketal (6)

Compound 5 (124 mg, 0.276 mmol) was dissolved in anhydrous DMF (5 mL) and stirred under N₂. KI (137 mg, 0.828 mmol) was added, followed by cyclopropylmethyl bromide (0.134 mL, 1.38 mmol). The resulting mixture was heated at 80-90° C. for 18 h. More cyclopropylmethyl bromide (0.10 mL, 1.03 mmol) was added and the reaction was continued for another 24 h. The reaction solution was concentrated and the residue was purified by column (eluent: 5-10% MeOH in DCM) to give 6 (123 mg, 75%) as a yellow solid. ¹H NMR (300 MHz, DMSO-d₆) δ ppm 7.95 (s, 1H), 7.28-7.44 (m, 5H), 6.92 (d, J=8.5 Hz, 1H), 6.68 (d, J=8.5 Hz, 1H), 5.11 (s, 2H), 4.99 (s, 1H), 4.71 (d, J=5.0 Hz, 1H), 4.62 (d, J=6.6 Hz, 1H), 3.86-4.27 (m, 3H), 3.45 (s, 3H), 3.28-3.76 (m, 4H), 3.13-3.27 (m, 1H), 2.21-2.33 (m, 1H), 1.74-1.93 (m, 2H), 1.12-1.40 (m, 4H), 0.73 (d, J=8.0 Hz, 2H), 0.48 (d, J=2.2 Hz, 2H). MS [M+H]: 504.3

### (vi) (S)-17-Cyclopropylmethyl-4,5α-epoxy-3-hydroxy-(17,14-N,O-ethylene)morphinanium-6-one trifluoroacetate (D0007)

Compound 6 (121 mg, 0.21 mmol) was dissolved in TFA (5 mL) and heated at 60-70° C. for 4.5 h. TFA was removed. The brown solid residue was combined with the crude product from another reaction (67 mg of 2) and purified by semi-prep HPLC to give D0007 (71 mg, TFA salt, 46%) as a white foam. ¹H NMR (300 MHz, D₂O) δ ppm 6.79 (d, J=8.3 Hz, 1H), 6.72 (d, J=8.5 Hz, 1H), 5.06 (s, 1H), 4.64 (d, J=5.5 Hz, 1H), 4.27-4.41 (m, 1H), 4.09-4.27 (m, 2H), 3.88-4.04 (m, 1H), 3.45-3.75 (m, 3H), 3.12-3.29 (m, 2H), 2.92-3.10 (m, 3H), 2.09-2.29 (m, 2H), 1.80 (d, J=9.6 Hz, 1H), 1.50-1.68 (m, 1H), 1.06-1.23 (m, 1H), 0.69-0.89 (m, 2H), 0.35-0.55 (m, 2H). HPLC purity: 100%. MS [M+H]: 368.1.

### EXAMPLES 8-11

### 17-Cyclopropylmethyl-4,5α-epoxy-3,14-dihydroxy-17-methylmorphinanium triflate (18) (D0008), 17-Cyclopropylmethyl-4,5α-epoxy-3-hydroxy-17-methyl-14-(3-phenylpropyloxy) morphinanium triflate (21) (D0009),

### 17-Cyclopropylmethyl-4,5α-epoxy-3-hydroxy-17-methyl-14-propyloxy morphinanium triflate (20) (D0010), and 17-cyclopropylmethyl-4,5α-epoxy-3-hydroxy-14-methoxy-17-methyl morphinanium triflate (19) (D0011)

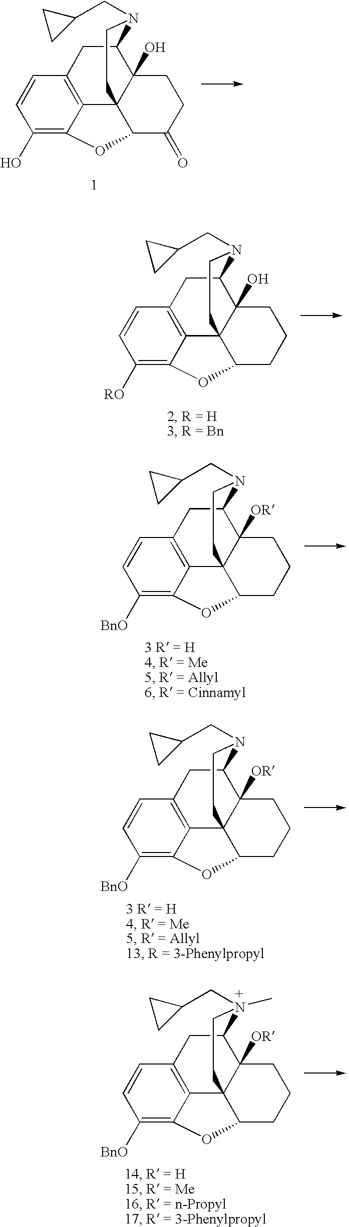

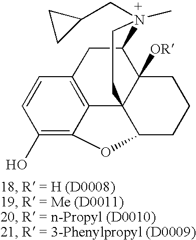

### General procedure for the 3-O-benzylation of 17-cyclopropylmethyl-4,5α-epoxy-3,14-dihydroxymorphinan derivatives

To a solution of the 3-hydroxy compound (1 eq.) in DMF (2 mL/mmol) under N₂ was added K₂CO₃ (1.3 eq) followed by benzyl bromide (1.1 eq) and the resulting mixture stirred for 20 h. The reaction mixture was diluted with water and extracted with dichloromethane. The combined organics were dried over MgSO₄ and concentrated to give the crude 3-O-benzyl derivative that was further treated as described in the individual cases.

### General procedure for the 14-O-alkylation of 3-Benzyloxy-17-cyclopropylmethyl-4,5α-epoxy-14-hydroxymorphinan derivatives

NaH (3 eq, 60% suspension in mineral oil) was added to a solution of 3-benzyloxy-17-cyclopropylmethyl-4,5α-epoxy-14-hydroxymorphinan derivatives (1 eq.) in DMF under N₂. After 20 minutes the alkyl halide/alkyl sulfate was added (1.3 eq.) and the resulting mixture was stirred for 2-5 h at room temperature. Excess NaH was destroyed by the addition of ice. Water was added and the reaction mixture was extracted with dichloromethane. The organics were pooled and dried (MgSO₄) and evaporated to provide the crude material that was purified whenever necessary or used as such further.

### General Procedure for Hydrogenation:

10-50 Mol % of the palladium catalyst (10% Pd on carbon, 50% wet) was added to a solution of the compound in methanol or methanol-THF mixture (1:1) and hydrogenated at 1 atmosphere pressure for 2 to 3 h at room temperature. The catalyst was filtered off and the filtrate was evaporated to give the crude product which was used as such without further purification for the next step.

### 17-Cyclopropylmethyl-4,5α-epoxy-3,14-dihydroxy-17-methylmorphinanium triflate (18) (D0008)

3-Benzyloxy-14-cinnamyloxy-17-cyclopropylmethyl-4,5α-epoxymorphinan 6 was synthesised from 3-benzyloxy-17-cyclopropylmethyl-4,5α-epoxy-14-hydroxymorphinan 3 by treating with cinnamyl bromide and NaH as described in the general procedure (Yield=62%).

¹H NMR (301 MHz, CHLOROFORM-d) δ ppm 7.38-7.46 (m, 5H), 6.73 (d, J=8.3 Hz, 1H), 6.66 (d, J=16 Hz, 1H), 6.54 (d, J=8.0 Hz, 1H), 6.29-6.47 (m, 1H), 5.16 (dd, J=15.7, 12.1 Hz, 2H), 4.78 (t, J=7.4 Hz, 1H), 4.38 (dd, J=5.0, 1.4 Hz, 1H), 4.34 (dd, J=5.5, 1.7 Hz, 1H), 3.94-4.05 (m, 1H), 3.44 (d, J=5.0 Hz, 1H), 3.11 (d, J=18.4 Hz, 1H), 2.63-2.77 (m, 1H), 2.47-2.62 (m, 1H), 2.43 (d, J=5.5 Hz, 1H), 2.31-2.39 (m, 5H), 2.05-2.22 (m, 4H), 1.68-1.77 (m, 2H), 1.30-1.39 (m, 1H), 0.99-1.17 (m, 1H), 0.76-0.91 (m, 1H), 0.34-0.59 (m, 2H), 0.13 (d, J=5.0 Hz, 2H); APCI [M+H] 534.3.

### (ii) 3-benzyloxy-17-cyclopropylmethyl-4,5α-epoxy-14-hydroxymorphinan

A solution of 3-benzyloxy-14-cinnamyloxy-17-cyclopropylmethyl-4,5-epoxymorphinan 6 (1 eq), in dichloromethane was treated with methyl triflate (3 eq. 0.69 M solution in dichloromethane) at room temperature for 36 h. The solvent was evaporated and the residue purified by silica column chromatography (Dichloromethane/Methanol) to afford 3-benzyloxy-17-cyclopropylmethyl-4,5α-epoxy-14-hydroxymorphinan 14 (MeOTf cleaves the 14-O-cinnamyl ether) (95%).

¹H NMR (301 MHz, CHLOROFORM-d) δ ppm 7.30-7.39 (m, 5H), 6.84 (d, J=8.25 Hz, 1H), 6.67 (d, J=8.25.0 Hz, 1H), 5.16 (s, 2H), 4.78 (t, J=7.4 Hz, 1H), 4.69 (br. s. 1H), 4.25 (d, J=3.3 Hz, 1H), 4.79 (m, 4H), 3.36-3.47 (m, 2H), 3.10-3.22 (m, 2H), 2.59-2.88 (m, 3H), 2.11-2.16 (m, 1H), 1.64 (d, J=13.17 Hz, 1H), 1.15-1.39 (m, 5H), 0.82-0.877 (m, 2H), 0.59-0.62 (m, 1H), 0.36-0.41 (m, 1H) APCI [M+H] 432.3.

### 17-Cyclopropylmethyl-4,5α-epoxy-3,14-dihydroxy-17-methylmorphinanium triflate (18) (D0008)

Hydrogenation of 14 as described in the general procedure afforded the title compound D0008 in 98% yield.

¹H NMR (301 MHz, DEUTERIUM OXIDE) □ ppm 6.82 (d, J=8.3 Hz, 1H), 6.77 (d, J=8.0 Hz, 1H), 4.85 (t, J=7.4 Hz, 1H), 3.80-4.02 (m, 2H), 3.53-3.66 (m, 4H), 3.12-3.34 (m, 3H), 2.90-3.04 (m, 1H), 2.63-2.74 (m, 1H), 2.44-2.62 (m, 1H), 2.06-2.20 (m, 1H), 1.54-1.75 (m, 2H), 1.35-1.50 (m, 2H), 1.07-1.25 (m, 2H), 0.68-0.93 (m, 2H), 0.49-0.61 (m, 1H), 0.30-0.40 (m, 1H); APCI [M+H] 342.2; HPLC (85/15 Water/Methanol with 0.1% TFA) R_{T}=5.49 min.

### 17-Cyclopropylmethyl-4,5α-epoxy-3-hydroxy-17-methyl-14-(3-phenylpropyloxy)morphinanium triflate (21) (D0009)

### (i) 17-cyclopropylmethyl-4,5α-epoxy-3-hydroxy-14-(3-phenylpropyloxy)morphinan

3-Benzyloxy-14-cinnamyloxy-17-cyclopropylmethyl-4,5α-epoxymorphinan (6) was hydrogenated as described in the general procedure to afford 17-cyclopropylmethyl-4,5α-epoxy-3-hydroxy-14-(3-phenylpropyloxy)morphinan (9) in quantitative yield.

¹H NMR (301 MHz, CHLOROFORM-d) δ ppm 6.99-7.44 (m, 5H), 6.70 (d, J=8.0 Hz, 1H), 6.55 (d, J=8.0 Hz, 1H), 4.75 (t, J=7.4 Hz, 1H), 3.59-3.70 (m, 2H), 3.22-3.45 (m, 2H), 3.09 (d, J=17.9 Hz, 1H), 2.78 (t, J=8.0, 7.4 Hz, 2H), 2.47-2.73 (m, 4H), 2.27-2.43 (m, 4H), 2.04-2.19 (m, 1H), 1.89-2.02 (m, 2H), 1.69 (d, J=12.9 Hz, 1H), 1.19-1.35 (m, 2H), 0.93-1.13 (m, 1H), 0.68-0.85 (m, 1H), 0.42-0.44 (m, 2H), 0.04-0.17 (m, 2H); APCI [M+H] 446.3.

### (ii) 3-benzyloxy-17-cyclopropylmethyl-4,5α-epoxy-14-(3-phenylpropyloxy)morphinan

A solution of 9 in DMF under N₂ was treated with K₂CO₃ and benzyl bromide for 20 h at room temperature. The reaction mixture was diluted with water and extracted with dichloromethane. The combined organics were dried over MgSO₄ and concentrated the solvent to yield 3-benzyloxy-17-cyclopropylmethyl-4,5′-epoxy-14-(3-phenylpropyloxy)morphinan 13 after purification by silica column chromatography using hexane/ethylacetate as eluent in 68% yield.

¹H NMR (301 MHz, CHLOROFORM-d) δ ppm 7.11-7.55 (m, 10H), 6.72 (d, J=7.4 Hz, 1H), 6.52 (d, J=8.0 Hz, 1H), 5.15 (m, 2H), 4.72 (t, J=7.7 Hz, 1H), 3.54-3.67 (m, 1H), 3.35 (d, J=4.7 Hz, 1H), 3.23-3.31 (m, 1H), 3.07 (d, J=18.4 Hz, 1H), 2.78 (t, J=7.7 Hz, 2H), 2.65 (dd, J=11.6, 4.7 Hz, 1H), 2.44-2.59 (m, 1H), 2.25-2.41 (m, 2H), 2.00-2.20 (m, 2H), 1.82-1.98 (m, 2H), 1.61-1.74 (m, 2H), 1.15-1.45 (m, 4H), 0.93-1.11 (m, 0H), 0.63-0.80 (m, 1H), 0.39-0.52 (m, 2H), 0.02-0.10 (m, 2H); APCI [M+H] 536.2

### (iii) 3-benzyloxy-17-cyclopropylmethyl-4,5α-epoxy-17-methyl-14-(3-phenylpropyloxy)morphinanium triflate

A solution of 13 (1 eq) in dichloromethane was treated with methyl triflate (3 eq. 0.69 M solution in dichloromethane) at room temperature for 17 h under N₂ atmosphere. The solvent was evaporated and the crude material was purified by preparative TLC (1000 V plate, eluent MeOH/DCM, 5/95) to afford 55% of 3-benzyloxy-17-cyclopropylmethyl-4,5α-epoxy-17-methyl-14-(3-phenylpropyloxy) morphinanium triflate (17) as white solid.

¹H NMR (301 MHz, CHLOROFORM-d) δ ppm 7.16-7.48 (m, 10H), 6.84 (d, J=8.5 Hz, 1H), 6.68 (d, J=8.0 Hz, 1H), 5.16 (s, 2H), 4.66 (t, t, J=8.0, 7.4 Hz, 1H), 4.50 (s, 1H), 3.69-3.85 (m, 2H), 3.63 (s, 3H), 3.42-3.58 (m, 2H), 3.20-3.38 (m, 2H), 2.91-3.16 (m, 2H), 2.69-2.91 (m, 2H), 2.44-2.64 (m, 1H), 1.89-2.25 (m, 5H), 1.58-1.72 (m, 1H), 1.32 (m, 2H), 0.99-1.27 (m, 2H), 0.77-0.99 (m, 2H), 0.48-0.65 (m, 2H); APCI [M+H] 550.4.

### (iv) 17-Cyclopropylmethyl-4,5α-epoxy-3-hydroxy-17-methyl-14-(3-phenylpropyloxy)morphinanium triflate

A methanolic solution of 17 was subjected to hydrogenation as described in the general procedure to afford the title compound 21, D0009, in quantitative yield.

¹H NMR (301 MHz, CHLOROFORM-d) δ ppm 9.37 (s, 1H), 7.04-7.41 (m, 5H), 6.42-6.92 (m, 2H), 4.66 (t, J=8.0, 7.4 Hz, 1H), 4.27-4.32 (m, 1H), 3.70-3.84 (m, 1H), 3.51-3.65 (m, 2H), 3.46 (s, 3H), 3.39 (d, J=1.9 Hz, 1H), 3.14-3.26 (m, 1H), 2.80-3.01 (m, 3H), 2.62-2.80 (m, 4H), 1.78-2.19 (m, 4H), 1.47 (d, J=14.0 Hz, 1H), 0.98-1.31 (m, 4H), 0.45-0.88 (m, 2H), 0.14-0.42 (m, 1H); APCI [M+H] 460.3; HPLC (40/60 Water/Methanol with 0.1% TFA) R_{T}=4.50 min.

### 17-Cyclopropylmethyl-4,5α-epoxy-3-hydroxy-17-methyl-14-propyloxy morphinanium triflate (20) (D0010)

### (i) 14-allyloxy-3-benzyloxy-17-cyclopropylmethyl-4,5α-epoxy-17-methylmorphinan

A solution of 14-allyloxy-3-benzyloxy-17-cyclopropylmethyl-4,5α-epoxymorphinan 5 (1 eq) in dichloromethane was treated with methyl triflate (3 eq. 0.69 M solution in dichloromethane) at room temperature for 17 h. The solvent was evaporated and the residue purified by silica column chromatography (Dichloromethane/Methanol) to afford 14-allyloxy-3-benzyloxy-17-cyclopropylmethyl-4,5α-epoxy-17-methylmorphinan 16 in 76% yield which was slightly impure by NMR and was used as such for the next step.

¹H NMR (301 MHz, CHLOROFORM-d) δ ppm 7.26-7.46 (m, 5H), 6.84 (d, J=8.3 Hz, 1H), 6.69 (d, J=8.3 Hz, 1H), 5.82-6.03 (m, 1H), 5.38 (d, J=17.1 Hz, 1H), 5.21 (d, J=10.5 Hz, 1H), 5.11 (s, 2H), 4.78 (d, J=8.1 Hz, 1H), 4.58 (br. s., 1H), 4.14-4.30 (m, 1H), 3.97-4.10 (m, 1H), 3.73 (dd, 1H), 3.59 (s, 3H), 3.55 (br. s., 1H), 3.47 (d, J=5.2 Hz, 1H), 3.34 (d, J=10.7 Hz, 1H), 3.10 (dd, J=20.4, 3.6 Hz, 1H), 2.61-2.99 (m, 2H), 2.05-2.29 (m, 2H), 1.68 (d, J=13.5 Hz, 1H), 1.33-1.49 (m, 3H), 1.14-1.29 (m, 2H), 0.71-1.01 (m, 2H), 0.25-0.66 (m, 2H); APCI [M+H] 472.3.

### (ii) 17-Cyclopropylmethyl-4,5α-epoxy-3-hydroxy-17-methyl-14-propyloxy morphinanium triflate (20) (D0010)

Hydrogenation of 16 was performed as as described in the general procedure. The crude material was purified on a semi-prep HPLC column using methanol/water (30/70) with 0.1% TFA and afforded the title compound D0010 as a white solid (32%).

¹H NMR (301 MHz, METHANOL-d₃) δ ppm 6.63-6.82 (m, 2H), 4.74 (t, J=8.3, 7.7 Hz, 1H), 4.29 (d, J=3.6Hz, 1H) δ 3.88 (dd, J=14.0, 3.9 Hz, 1H), 3.64 (d, J=1.4 Hz, 1H), 3.61 (s, 3H), 3.52-3.58 (m, 2H), 3.40-3.48 (m, 1H), 3.04-3.12 (m, 3H), 2.95-3.04 (m, 1H), 2.68-2.81 (m, 2H), 2.12-2.25 (m, 1H), 2.02-2.11 (m, 1H), 1.62-1.78 (m, 2H), 1.48-1.56 (m, 1H), 1.41-1.47 (m, 1H), 1.20-1.34 (m, 2H), 1.03 (t, J=7.4 Hz, 3H), 0.87-0.99 (m, 1H), 0.76-0.88 (m, 1H), 0.59-0.69 (m, 1H), 0.37-0.48 (m, 1H); APCI [M+H] 384.3; HPLC (50/50 Water/Methanol with 0.1% TFA) R_{T}=4.30 min.

### 17-cyclopropylmethyl-4,5α-epoxy-3-hydroxy-14-methoxy-17-methyl morphinanium triflate (19) (D0011)

### (i) 3-Hydroxy-17-cyclopropylmethyl-4,5α-epoxy-14-hydroxymorphinan (2)

To a solution of naltrexone hydrochloride (1.HCl, 10 g, 1 eq.) in diethyleneglycol (55 mL) was added hydrazine hydrate (98%, 8 mL) and potassium hydroxide (28 g, 30 eq.) and the mixture was heated at 100° C. for 1 h and 165° C. for 3 h. The reaction mixture was cooled and diluted with water (150 mL) and acidified to pH 6 with conc. HCl and then to pH 10 with solid NaHCO₃ and extracted with methanol:dichloromethane (1:9) (2×200 mL). The combined organics were dried over MgSO₄ and concentrated to get a brownish residue. Purification of the crude material by column chromatography using hexanes/ethylacetate/triethylamine; 50/45/5 afforded 4.5 g (35%) of the 17-cyclopropylmethyl-4,5α-epoxy-3,14-dihydroxymorphinan 2 as a white solid.

¹H NMR (301 MHz, CHLOROFORM-d) δ ppm 6.69 (d, J=8.0 Hz, 1H), 6.51-6.62 (d, J=8.0 Hz, 1H), 5.13 (br. s., 1H), 4.73 (t, J=8.22 Hz, 1H), 2.95-3.08 (m, 2H), 2.54-2.70 (m, 2H), 2.31-2.41 (m, 2H), 2.06-2.27 (m, 2H), 1.77 (tt, J=12.9, 3.0 Hz, 1H), 1.35-1.57 (m, 2H), 1.13-1.35 (m, 2H), 0.74-0.94 (m, 1H), 0.45-0.59 (m, 2H), 0.41-0.70 (m, 2H), 0.07-0.23 (m, 2H); APCI [M+H] 328.2.

### (ii) 3-Benzyloxy-17-cyclopropylmethyl-4,5α-epoxy-14-hydroxymorphinan (3)

A solution of 2 (0.991 g, 1 eq.) in DMF (10 mL) under N₂ was treated with K₂CO₃ and benzyl bromide as described in the general procedure. The mixture was stirred for 20 h. The reaction mixture was diluted with water and extracted with dichloromethane. The combined organics were dried over MgSO₄ and the solvent concentrated to provide 1.2 g (95%) of the title compound 3 which was used for the next step without further purification.

¹H NMR (301 MHz, CHLOROFORM-d) δ ppm 7.29-7.47 (m, 5H), 6.75 (d, J=8.3 Hz, 1H), 6.56 (d, J=8.0 Hz, 1H), 5.17 (dd, J=26.4, 12.4 Hz, 2H), 5.08 (s, 1H), 4.73 (t, J=8.0 Hz, 1H), 2.94-3.12 (m, 2H), 2.51-2.73 (m, 2H), 2.36 (d, J=6.6 Hz, 2H), 2.03-2.28 (m, 3H), 1.67-1.99 (m, 1H), 1.10-1.65 (m, 5H), 0.72-0.92 (m, 1H), 0.53 (d, J=7.7 Hz, 2H), 0.00-0.26 (m, 2H); APCI [M+H] 418.3.

### (iii) 17-cyclopropylmethyl-4,5α-epoxy-3-hydroxy-14-methoxy-17-methylmorphinan

A solution of 3-benzyloxy-17-cyclopropylmethyl-4,5α-epoxy-14-methoxymorphinan (4) (1 eq), in dichloromethane was treated with methyl triflate (3 eq. 0.69 M solution in dichloromethane) at room temperature for 17 h. The solvent was evaporated and the residue purified by silica column chromatography (Dichloromethane/Methanol) to afford 17-cyclopropylmethyl-4,5α-epoxy-3-hydroxy-14-methoxy-17-methylmorphinan 15 in 90% yield.

¹H NMR (301 MHz, CHLOROFORM-d) δ ppm 7.21-7.42 (m, 5H), 6.83 (d, J=8.3 Hz, 1H), 6.68 (d, J=8.3 Hz, 1H), 5.14 (dd, J=14.0, 12.1 Hz, 2H), 4.72 (t, J=8.0 Hz, 1H), 4.45 (d, J=2.5 Hz, 1H), 3.72 (dd, J=13.5, 4.1 Hz, 1H), 3.60 (s, 3H), 3.49 (d, J=20.4 Hz, 1H), 3.32-3.39 (m, 2H), 3.25 (s, 1H), 2.96-3.15 (m, 2H), 2.56-2.73 (m, 2H), 2.30-2.51 (m, 1H), 2.05-2.25 (m, 3H), 1.54-1.66 (m, 1H), 1.24-1.49 (m, 3H), 0.88-1.01 (m, 1H), 0.69-0.85 (m, 1H), 0.38-0.67 (m, 2H); APCI [M+H] 446.2

### (iv) 17-cyclopropylmethyl-4,5α-epoxy-3-hydroxy-14-methoxy-17-methyl morphinanium triflate (19) (D001)

Hydrogenation of 15 was performed as as described in the general procedure. The crude material was purified on a semi-prep HPLC column using methanol/water (35/85) with 0.1% TFA afforded the title compound D0011 as a white solid (77%).

¹H NMR (301 MHz, DEUTERIUM OXIDE) δ ppm 6.79 (d, J=8.3 Hz, 1H), 6.71 (d, J=8.3 Hz, 1H), 4.80 (t, J=8.3 Hz, 1H), 4.24 (d, J=3.6 Hz, 1H), 3.79-3.97 (m, 1H), 3.59 (d, J=19.8 Hz, 1H), 3.49 (s, 3H), 3.26 (s, 3H), 3.12-3.22 (m, 1H), 2.82-3.09 (m, 2H), 2.49-2.71 (m, 2H), 2.02-2.22 (m, 1H), 1.94 (d, J=14.0 Hz, 1H), 1.59 (dd, J=14.9, 3.3 Hz, 1H), 1.31-1.45 (m, 2H), 1.04-1.25 (m, 3H), 0.63-0.92 (m, 2H), 0.46-0.59 (m, 1H), 0.18-0.43 (m, 1H); APCI [M+H] 356.3; HPLC (75/25 Water/Methanol with 0.1% TFA) R_{T}=5.20 min.

### Statement Regarding Embodiments

While the invention has been described with respect to embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

1. Compounds having the formula I(e): 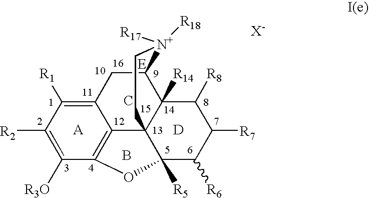
or a pharmaceutically acceptable salt form, polymorph, or prodrug thereof, wherein:
R₁ and R₂ are independently H, OH, OR₂₉, halide, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₁ and R₂ are combined to form a C₃-C₆ carbocycle fused ring, a benzo fused ring,
or a 5-6 membered heteroaryl fused ring;
R₃ is H, silyl, CO₂R₁₉, SO₂R₁₉, B(OR₁₉)₂;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₅ is H, OH, OR₂₉,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, N(CH₃)₂, ═(R₁₉)(R_{19′}), =(hetero cycle substituted with 0-3R₂₀), ═(C₃-C₇ cycle substituted with 0-3R₂₀) or any cyclic ring;
R₇ is H, OH, OR₂₉,
(C₁-C₂₀) alkyl substituted with 0-3 R₁₉;
(C₂-C₂₀) alkenyl substituted with 0-3 R₁₉;
(C₂-C₂₀) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₆ and R₇ are combined to form an O-fused ring, a C₃-C₆ carbocycle fused ring, a benzo fused ring, 5-, 6- or a 5-6 membered aryl with 0-3 R₂₀, or a heteroaryl fused ring;
R₈ is H, OH, OR₂₉, hetero cycle with 0-3R₂₀, alkylaryl with 0-3R₂₀, arylalkyl with 0-3 R₂₀, 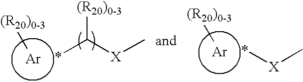
wherein, X is bond, ═O, O, S, N(R₂₉), SO—SO₂, SO₂N(R₂₉), CON(R₂₉), N(R₂₉)CON(R_{29′}), N(R₂₉)C(═NR_{29′})N(R_{29″}), COO,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₁₄ is H, OH, halide, hetero cycle with 0-3R₂₀, alkylaryl with 0-3R₂₀, arylalkyl with 0-3 R₂₀, 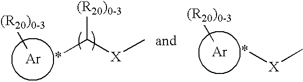
wherein, X is bond, ═O, O, S, N(R₂₉), SO, SO₂, SO₂N(R₂₉), CON(R₂₉), N(R₂₉)CON(R_{29′}), N(R₂₉)C(═NR_{29′})N(R_{29″}), COO,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or combined with R₁₈ to form an O-fused ring, or a C₃-C₆ carbocycle fused ring, or if R₆=a cyclic ring, or forms a cyclic ring with R₇, may be further be an alkoxy or aryloxy;
wherein if R₆ is ═O, R₁₄ is not:
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
R₁₇ is heterocycle with 0-3R₂₀, alkylaryl with 0-3R₂₀, arylalkyl with 0-3 R₂₀, 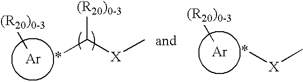
wherein, X is bond, ═O, O, S, N(R₂₉), SO, SO₂, SO₂N(R₂₉), CON(R₂₉), N(R₂₉)CON(R_{29′}), N(R₂₉)C(═NR_{29′})N(R_{29″}), COO,
(C₄-C₂₀) alkyl substituted with 0-3 R₂₅;
(C₄-C₂₀) alkenyl substituted with 0-3 R₂₅;
(C₄-C₂₀) alkynyl substituted with 0-3 R₂₅;
(C₃-C₁₀) cycloalkyl substituted with 0-3R₂₆;
(C₃-C₁₀) carbocycle substituted with 0-3R₂₆;
aryl substituted with 0-3R₂₆;
R₁₈ is (C₁-C₃) alkyl substituted with 0-3 R₂₇;
(C₂-C₄) alkenyl substituted with 0-3 R₂₇;
(C₂-C₄) alkynyl substituted with 0-3 R₂₇;
R₁₉ is at each occurrence is independently selected from:
H, aryl substituted with 0-3R₂₀, C₁-C₆ alkyl, CF₃, OR₂₄, Cl, F, Br, I, ═O, CN, NO₂, NR₂₂R₂₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl, OR₂₅, XR₂₅,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl, OR₂₅, XR₂₅.
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, C₆-C₁₀ aryl, hetero aryl, hetero cycle, alkylaryl, arylalkyl,
(C₁-C₆ alkyl)-C(═O)—, and (C₁-C₆ alkyl)-S(═O)₂—;
R₂₃, at each occurrence, is independently selected from:
H, (C₁-C₆) alkyl, C₆-C₁₀ aryl, hetero aryl, hetero cycle, alkylaryl, haloalkyl, and arylalkyl,
(C₁-C₆ alkyl)-C(═O)—, and (C₁-C₆ alkyl)-S(═O)₂—;
wherein R₂₂ and R₂₃ may further be combined to form 5-, 6-, 5-6-membered cycle with 0-3R₂₀;
R₂₄, at each occurrence, is independently selected from H, phenyl, benzyl, (C₁-C₆) alkyl, haloalkyl and (C₂-C₆) alkoxyalkyl;
R₂₅, at each occurrence, is independently selected from:
H, C₁-C₆ alkyl, haloalkyl, OR₂₄, ═O, CN, NO₂, NR₂₇R₂₈;
C₃-C₁₀ carbocycle substituted with 0-3 R₂₇;
aryl substituted with 0-3 R₂₇; or
5 to 10 membered heterocycle containing 1 to 4 heteroatoms selected from nitrogen, oxygen, wherein said 5 to 10 membered heterocycle is substituted with 0-3 R₂₇;
R₂₆, at each occurrence, is independently selected from:
H, (C₁-C₆)alkyl, benzyl, phenyl, phenethyl, (C₁-C₆ alkyl)-C(═O)—;
R₂₇, at each occurrence, is independently selected from:
—OH, —OR₂₈, C₁-C₆ alkyl, C₁-C₄ alkoxy;
R₂₈, at each occurrence, is independently selected from:
C₁-C₆ alkyl; (C₁-C₆ alkyl)-C(═O)—, and (C₁-C₆ alkyl)-S(—O)₂—; and
R₂₉ is at each occurrence is independently selected from:
H, C₁-C₆ alkyl, CF₃, acyl(C₁-C₆)alkyl;
acylaryl substituted with 0-3 R₂₁;
C₃-C₁₀ carbocycle substituted with 0-3 R₂₁;
aralkyl substituted with 0-3 R₂₁;
5 to 10 membered heterocycle containing 1 to 4 heteroatoms selected from nitrogen, oxygen, and sulphur, wherein said 5 to 10 membered heterocycle is substituted with 0-3 R₂₁; or aryl substituted with 0-3R₂₀; and
X⁻ is an anion

2. Compounds having the formula I: 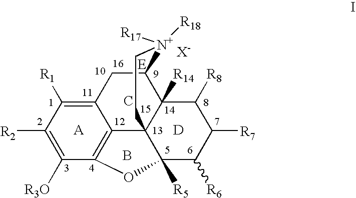
or a pharmaceutically acceptable salt form, polymorph, or prodrug thereof, wherein:
R₁₇ and R₁₈ are selected alternatively with respect to one another from (a) or (b):
(a) unsubstituted or non-halogen substituted: C4-C20 (cycloalkyl)alkyl or (cycloalkenyl)alkyl, (cycloheteryl)alkyl, (cycloaryl)alkyl; C4-C10 (cycloalkyl)alkyl or (cycloalkenyl)alkyl, (cycloheteryl)alkyl, (cycloaryl)alkyl
(b) substituted or unsubstituted linear or branched C1-C3 alkyl, C2-C3 alkenyl, or C3 alkynyl;
wherein if (b) is selected as methyl, and R6 below is selected as ═O, (a) is not unsubstituted (cyclopropyl)methyl;
R₆ is O, ═CH₂, —N(CH₃)₂, or any cyclic ring, or forms a cyclic ring with R₇;
R₇ and R₈ are H or alkyl;
R₁₄ is OH, halide, amido, amino, or forms a cyclic ring with R₁₈, and if R₆=a cyclic ring, or forms a cyclic ring with R₇, may further be an alkoxy or aryloxy, and if R₆ is not ═O, R₁₄ may be alkoxy or aryloxy;
R₁ and R₂ are independently H, halide, alkoxy, alkyl, or aryl
R₃ is H, C₁-C₄ alkyl, or C1-C3 acyl, -silyl
R₅ is H, OH, alkyl, alkoxy, or aryloxy; and
X⁻ is an anion.

3. Compounds having the formula I(a): 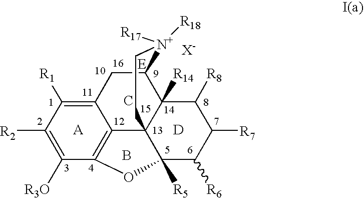
or a pharmaceutically acceptable salt form or prodrug thereof, wherein:
R₁ and R₂ are independently H, OH, OR₂₉, halide, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₁ and R₂ are combined to form a C₃-C₆ carbocycle fused ring, a benzo fused ring,
or a 5-6 membered heteroaryl fused ring;
R₃ is H, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₅ is H, OH, OR₂₉,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O, N(CH₃)₂, or any cyclic ring;
R₇ is H, OH, OR₂₉,
(C₁-C₂₀) alkyl substituted with 0-3 R₁₉;
(C₂-C₂₀) alkenyl substituted with 0-3 R₁₉;
(C₂-C₂₀) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₆ and R₇ are combined to form an O-fused ring, a C₃-C₆ carbocycle fused ring, a benzo fused ring, or a 5-6 membered heteroaryl fused ring;
R₈ is H, OH, OR₂₉
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₁₄ is H, OH, halide,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or combined with R₁₈ to form an O-fused ring, or a C₃-C₆ carbocycle fused ring, or if R₆=a cyclic ring, or forms a cyclic ring with R₇, may be further be an alkoxy or aryloxy;
wherein if R₆ is ═O, R₁₄ is not:
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
R₁₇ is (C₄-C₂₀) alkyl substituted with 0-3 R₂₅;
(C₄-C₂₀) alkenyl substituted with 0-3 R₂₅;
(C₄-C₂₀) alkynyl substituted with 0-3 R₂₅;
(C₃-C₁₀) cycloalkyl substituted with 0-3R₂₆;
(C₃-C₁₀) carbocycle substituted with 0-3R₂₆;
aryl substituted with 0-3R₂₆;
R₁₈ is (C₁-C₃) alkyl substituted with 0-3 R₂₇;
(C₂-C₄) alkenyl substituted with 0-3 R₂₇;
(C₂-C₄) alkynyl substituted with 0-3 R₂₁;
R₁₉ is at each occurrence is independently selected from:
H, C₁-C₆ alkyl, CF₃, OR₂₄, Cl, F, Br, I, ═O, CN, NO₂, NR₂₂R₂₃;
C₃-C₁₀ carbocycle substituted with 0-3 R₂₁;
aryl substituted with 0-3 R₂₁; or
5 to 10 membered heterocycle containing 1 to 4 heteroatoms selected from nitrogen, oxygen, and sulphur, wherein said 5 to 10 membered heterocycle is substituted with 0-3 R₂₁;
R₂₀ at each occurrence, is independently selected from H, OH, Cl, F, Br, I, CN, NO₂, NR₂₂R₂₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, benzyl, phenethyl,
(C₁-C₆ alkyl)-C(═O)—, and (C₁-C₆ alkyl)-S(═O)₂—;
R₂₃, at each occurrence, is independently selected from:
H, (C₁-C₆) alkyl, benzyl, phenethyl,
(C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅, at each occurrence, is independently selected from:
H, C₁-C₆ alkyl, OR₂₄, ═O, CN, NO₂, NR₂, R₂₈;
C₃-C₁₀ carbocycle substituted with 0-3 R₂₇;
aryl substituted with 0-3 R₂₇; or
5 to 10 membered heterocycle containing 1 to 4 heteroatoms selected from nitrogen, oxygen, wherein said 5 to 10 membered heterocycle is substituted with 0-3 R₂₇;
R₂₆, at each occurrence, is independently selected from:
H, (C₁-C₆)alkyl, benzyl, phenyl, phenethyl, (C₁-C₆ alkyl)-C(═O)—;
R₂₇, at each occurrence, is independently selected from:
—OH, —OR₂₈, C₁-C₆ alkyl, C₁-C₄ alkoxy;
R₂₈, at each occurrence, is independently selected from:
C₁-C₆ alkyl; (C₁-C₆ alkyl)-C(═O)—, and (C₁-C₆ alkyl)-S(═O)₂—;
R₂₉ is at each occurrence is independently selected from:
H, C₁-C₆ alkyl, CF₃, acyl(C₁-C₆)alkyl;
acylaryl substituted with 0-3 R₂₁;
C₃-C₁₀ carbocycle substituted with 0-3 R₂₁;
aralkyl substituted with 0-3 R₂₁;
5 to 10 membered heterocycle containing 1 to 4 heteroatoms selected from nitrogen, oxygen, and sulphur, wherein said 5 to 10 membered heterocycle is substituted with 0-3 R₂₁; or aryl substituted with 0-3R₂₀; and
X⁻ is an anion.

4. Compounds having the formula I(b): 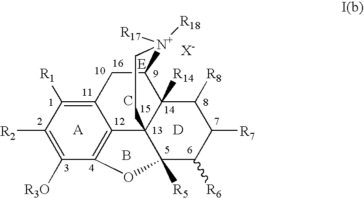
or a pharmaceutically acceptable salt form, polymorph, or prodrug thereof, wherein:
R₁ and R₂ are independently H, OH, OR₂₉, halide, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₁ and R₂ are combined to form a C₃-C₆ carbocycle fused ring, a benzo fused ring,
or a 5-6 membered heteroaryl fused ring;
R₃ is H, silyl;
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₅ is H, OH, OR₂₉,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₆ is H, ═O,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
amine, amide, sulfonamide, ester, heterocycle, cyclic carbohydride, aryl;
R₇ is H, OH, OR₂₉,
(C₁-C₂₀) alkyl substituted with 0-3 R₁₉;
(C₂-C₂₀) alkenyl substituted with 0-3 R₁₉;
(C₂-C₂₀) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
or R₆ and R₇ are combined to form an O-fused ring, a C₃-C₆ carbocycle fused ring, a benzo fused ring, or a 5-6 membered heteroaryl fused ring;
R₈ is H, OH, OR₂₉
(C₁-C₈) alkyl substituted with 0-3R₁₉,
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀;
R₁₄ is H, OH,
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
aryl substituted with 0-3R₂₀; aryloxy, acyloxy,
or R₁₄ is combined with R₁₈ to form an O-fused ring, or a C₃-C₆ carbocycle fused ring;
wherein if R₆=O, R₁₄ is not
(C₁-C₈) alkyl substituted with 0-3 R₁₉;
(C₂-C₈) alkenyl substituted with 0-3 R₁₉;
(C₂-C₈) alkynyl substituted with 0-3 R₁₉;
(C₃-C₁₀) cycloalkyl substituted with 0-3R₂₀;
(C₃-C₁₀) carbocycle substituted with 0-3R₂₀;
R₁₇ is (C₄-C₁₀) alkyl substituted with 0-3R₂₅;
(C₄-C₁₀) alkenyl substituted with 0-3R₂₅;
(C₄-C₁₀) alkynyl substituted with 0-3R₂₅;
(C₃-C₁₀) cycloalkyl substituted with 0-3R₂₆;
(C₃-C₁₀) carbocycle substituted with 0-3R₂₆;
aryl substituted with 0-3R₂₆;
R₁₈ is (C₁-C₃) alkyl substituted with 0-3R₂₇;
(C₂-C₄) alkenyl substituted with 0-3R₂₇;
(C₂-C₄) alkynyl substituted with 0-3R₂₇;
R₁₉ is at each occurrence is independently selected from:
H, C₁-C₆ alkyl, CF₃, OR₂₄, Cl, F, Br, I, ═O, CN, NO₂, NR₂₂R₂₃;
C₃-C₁₀ carbocycle substituted with 0-3R₂₁;
aryl substituted with 0-3R₂₁; or
a 5 to 10 membered heterocycle containing 1 to 4 heteroatoms selected from nitrogen, oxygen, and sulphur, wherein said 5 to 10 membered heterocycle is substituted with 0-3R₂₁;
R₂₀ at each occurrence, is independently selected from H, OH, Cl, F, Br, I, CN, NO₂, NR₂₂R₂₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—;
R₂₁, at each occurrence, is independently selected from H, OH, Cl, F, Br, I, CN, NO₂, NR₂₂R₂₃, CF₃, acetyl, SCH₃, S(═O)CH₃, S(═O)₂CH₃,
C₁-C₆ alkyl, C₁-C₄ alkoxy, C₁-C₄ haloalkyl,
C₁-C₄ haloalkoxy, and C₁-C₄ haloalkyl-S—; or
NR₂₂R₂₃ may be a heterocyclic ring selected from the group piperidinyl, homopiperidinyl, thiomorpholinyl, piperizinyl, and morpholinyl;
R₂₂, at each occurrence, is independently selected from H, C₁-C₆ alkyl, benzyl, phenethyl,
(C₁-C₆ alkyl)-C(═O)—, and (C₁-C₆ alkyl)-S(═O)₂—;
R₂₃, at each occurrence, is independently selected from:
H, (C₁-C₆) alkyl, benzyl, phenethyl,
(C₁-C₆ alkyl)-C(═O)—, and (C₁-C₆ alkyl)-S(═O)₂—;
R₂₄, at each occurrence, is independently selected from H, phenyl, benzyl, (C₁-C₆) alkyl, and (C₂-C₆) alkoxyalkyl;
R₂₅, at each occurrence, is independently selected from:
H, C₁-C₆ alkyl, OR₂₄, ═O, CN, NO₂, NR₂₇R₂₈;
C₃-C₁₀ carbocycle substituted with 0-3R₂₇;
aryl substituted with 0-3R₂₇; or
a 5 to 10 membered heterocycle containing 1 to 4 heteroatoms selected from nitrogen, oxygen, wherein said 5 to 10 membered heterocycle is substituted with 0-3R₂₇;
R₂₆, at each occurrence, is independently selected from:
H, (C₁-C₆)alkyl, benzyl, phenyl, phenethyl, (C₁-C₆ alkyl)-C(═O)—;
R₂₇, at each occurrence, is independently selected from:
—OH, —OR₂₈, C₁-C₆ alkyl, C₁-C₄ alkoxy;
R₂₈, at each occurrence, is independently selected from:
C₁-C₆ alkyl; (C₁-C₆ alkyl)-C(═O)—, and (C₁-C₆ alkyl)-S(═O)₂—; and
R₂₉ is at each occurrence is independently selected from:
H, C₁-C₆ alkyl, CF₃, acyl(C₁-C₆)alkyl;
acylaryl substituted with 0-3R₂₁;
C₃-C₁₀ carbocycle substituted with 0-3R₂₁;
aralkyl substituted with 0-3R₂₁;
5 to 10 membered heterocycle containing 1 to 4 heteroatoms selected from nitrogen, oxygen, and sulphur, wherein said 5 to 10 membered heterocycle is substituted with 0-3R₂₁; or aryl substituted with 0-3R₂₀; and
X⁻ is an anion.

5. A composition comprising at least one compound, polymorph, or salt thereof, selected from the group consisting of:
17-cyclopropylmethyl-4,5α-epoxy-3,14-dihydroxy-17-methyl-6-methylenemorphinanium;
17-cyclopropylmethyl-4,5α-epoxy-14-hydroxy-17-methyl-3-propyloxy-6-oxomorphinanium;
17-Allyl-17-cyclopropylmethyl-4,5α-epoxy-3,14-dihydroxy-6-oxomorphinanium;
17-cyclobutylmethyl-4,5α-epoxy-3,14-dihydroxy-17-methyl-6-oxomorphinanium;
17-cyclopentylmethyl-4,5α-epoxy-3,14-dihydroxy-17-methyl-6-methylenemorphinanium;
17-(3,3′-dimethylallyl)-4,5α-epoxy-3,14-dihydroxy-17-methyl-6-oxomorphinanium;
17-(3′-phenylbut-2′-ynyl)-4,5α-epoxy-3,14-dihydroxy-17-methyl-6-oxomorphinanium;
17-(2′,2′-Difluorocyclopropyl)methyl-4,5α-epoxy-3,14-dihydroxy-17-methyl-6-oxomorphinanium;
17-cyclopropylmethyl-4,5α-epoxy-3-benzyloxy-14-hydroxy-17-methyl-6α-methoxy-morphinanium; and
17-cyclopropylmethyl-4,5α-epoxy-3,14-dihydroxy-17-methyl-6β-hydroxy-8-propoxy-morphinanium;
17-(2′-Methylcyclopropyl)methyl-4,5α-epoxy-3,14-dihydroxy-17-methyl-6-oxomorphinanium;
17-Cyclopropylmethyl-4,5α-epoxy-3,14-dihydroxy-17-methyl-6α-methoxy morphinanium;
17-Cyclopropylmethyl-4,5α-epoxy-3,14-dihydroxy-17-methyl-6β-methoxy morphinanium;
17-Cyclopropylmethyl-4,5α-epoxy-3-methoxy-14-hydroxy-17-methyl-6-methylenemorphinanium;
17-Cyclopropylmethyl-4,5α-epoxy-3,14-dihydroxy-17-methylmorphinanium;
3-Acetyl-17-cyclopropylmethyl-4,5α-epoxy-14-hydroxy-17-methylmorphinanium;
17-[(2′-tetrahydrofuryl)methyl]-4,5α-epoxy-3,14-dihydroxy-17-methyl-6-oxo-morphinaninium;
17-Cyclopropylmethyl-4,5α-epoxy-3-hydroxy-17-methyl-14-(3′-phenylpropyloxy)morphinanium;
17-Cyclopropylmethyl-4,5α-epoxy-3-hydroxy-17-methyl-14-propyloxy morphinanium;
17-Cyclopropylmethyl-4,5α-epoxy-3-hydroxy-17-methyl-14-methoxy-morphinanium;
17-methyl-4,5α-epoxy-3-hydroxy-(17,14-N,O-ethylene-6-oxo-morphinanium; and
17-Cyclopropylmethyl-4,5α-epoxy-3-hydroxy-(17,14-N,O-ethylene)-6-oxo-morphinanium.

6. A pharmaceutical composition comprising the compound of claim 5 and a pharmaceutically acceptable carrier.

7. The pharmaceutical composition of claim 6, wherein the pharmaceutical composition comprises an immediate release formulation, an enteric coating, a sustained release formulation or a lyophilized preparation.

8. The pharmaceutical composition of claim 7, wherein the pharmaceutical formulation is a packaged unit dosage.

9. The pharmaceutical composition of claim 8, wherein the packaged unit dosage is a solution.

10. The pharmaceutical composition of claim 6, further comprising an opioid.

11. The composition of claim 10, wherein the opioid is selected from the group consisting of alfentanil, anileridine, asimodiline, bremazocine, burprenorphine, butorphanol, codeine, dezocine, diacetylmorphine (heroin), dihydrocodeine, diphenyloxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sulfentanil, tilidine, trimebutine, tramadol, and combinations thereof.

12. The pharmaceutical composition of claim 6, further comprising at least one pharmaceutical agent that is not an opioid or an opioid antagonist.

13. The pharmaceutical composition of claim 12, wherein at least one pharmaceutical agent is a non-opioid analgesic/anti-pyretic, an antiviral agent, an anti-infective agent, an anticancer agent, an antispasmodic agent, an anti-muscarinic agent, an anti-inflammatory agent, a pro-motility agent, a 5HT₁ agonist, a 5HT₃ antagonist, a 5HT₄ antagonist, a 5HT₄ agonist, a bile salt sequestering agent, a bulk-forming agent, an alpha2-adrenergic agonist, a mineral oil, an antidepressant, a herbal medicine, an anti-emetic agent, an anti-diarrheal agent, a laxative, a stool softener, a fiber or a hematopoietic stimulating agent.

14. The composition of claim 13, wherein the anti-inflammatory agent is selected from the group consisting of non-steroidal anti-inflammatory drugs (NSAIDS), tumor necrosis factor inhibitors, basiliximab, daclizumab, infliximab, mycophenolate, mofetil, azothioprine, tacrolimus, steroids, sulfasalazine, olsalazine, mesalamine, and combinations thereof.

15. The pharmaceutical composition of claim 6 wherein the composition is an oral formulation.

16. The pharmaceutical composition of claim 6 wherein the composition is a lyophilized formulation or is a parenteral formulation.

17. The pharmaceutical composition of claim 6 wherein the composition is in a sustained release formulation.

18. A method for modulating mu-opioid receptors comprising administering to a patient in need of mu-opioid modulation the composition of claim 6 in a modulation effective amount.

19. The method of claim 18 wherein the mu-opioid receptor modulation is consistent with an opioid agonist.

20. The method of claim 18 wherein the mu-opioid receptor modulation is consistent with an opioid antagonist.

21. A method for modulating kappa or delta opioid receptors comprising administering to a patient in need of such modulation the composition of claim 6 in a modulation effective amount.

22. The method of claim 21 wherein the kappa modulation is consistent with a kappa agonist.

23. The method of claim 21 wherein the kappa modulation is consistent with a kappa antagonist.

24. The method of claim 21, wherein the delta modulation is consistent with a delta agonist.

25. The method of claim 21, wherein the delta modulation is consistent with a delta antagonist.

26. The method of claim 18 wherein the composition is subcutaneously administered.

27. The method of claim 18, wherein the composition is intravenously administered.

28. The method of claim 18 wherein the composition is administered orally.

29. Compounds having the formula I(c): 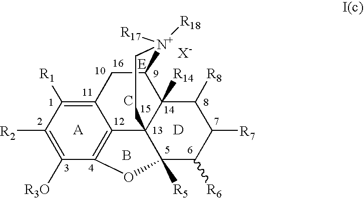
or a pharmaceutically acceptable salt form, polymorph, or prodrug thereof,
wherein:
R₁₇ and R₁₈ are selected alternatively with respect to one another from (a) or (b):
(a) unsubstituted or non-halogen substituted: C₄-C₂₀ (cycloalkyl)alkyl or (cycloalkenyl)alkyl, (cycloheteryl)alkyl, (cycloaryl)alkyl; C₄-C₁₀ (cycloalkyl)alkyl or (cycloalkenyl)alkyl, (cycloheteryl)alkyl, (cycloaryl)alkyl
(b) substituted or unsubstituted linear or branched C₁-C₃ alkyl, C₂-C₃ alkenyl, or C₃-alkynyl;
wherein if (b) is selected as methyl and R₆ below is selected ═O, (a) is not an unsubstituted (cyclopropyl)methyl;
R₆ is ═O, ═CH₂, —N(CH₃)₂, or any cyclic ring, or forms a cyclic ring with R₇;
R₇ and R₈ are H, hydrocarbyl, cyclohydrocarbyl, alkoxy, amine, amide, hydroxy or substituted moieties thereof;
R₁₄ is H, OH, halide, N-alkyl, N-dialkyl, N-aryl, N-alkylaryl, N-cycloalkylalkyl, or forms a cyclic ring with R₁₇ or R₁₈; and if R₆ is not —O, R₁₄ may be alkoxy, aryloxy, or aryl-alkoxy;
R₁ and R₂ are independently H, halide, alkoxy, alkyl, or aryl;
R₃ is H, C₁-C₄ alkyl, or C₁-C₃ acyl, -silyl;
R₅ is H, OH, alkyl, alkoxy, or aryloxy; and
X⁻ is an anion.

30. The compounds of claim 29 wherein, R₇ and R₈ are H or alkyl.

31. Compounds having the formula I(d): 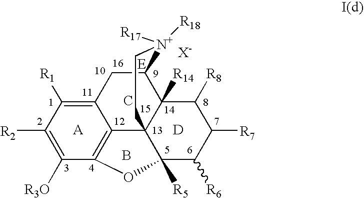
or a pharmaceutically acceptable salt form, polymorph, or prodrug thereof,
wherein:
R₁₇ and R₁₈ are a substituted or unsubstituted hydrocarbyl, when R6 is ═O at least one of which is not methyl when the other is unsubstituted cyclopropylmethyl;
R₆ is H, OH, OR₂₅, ═O, ═CH₂, —N-alkyl, N-dialkyl, acyloxy, alkoxy, alkyl, ═CR′R″ where R′ and R″ are independently H or C₁-C₁₀ alkyl, or any ring, or R₆ forms a ring with R₇;
R₇ and R₈ are H or hydrocarbyl, cyclohydrocarbyl, alkoxy, amine, amide, hydroxy or substituted moieties thereof,
R₁₄ is H, OH, halide, N-alkyl, N-dialkyl, N-aryl, N-alkylaryl, N-cycloalkylalkyl, SR₂₅, S(═O)R₂₅, SO₂R₂₅, or forms a cyclic ring with R₁₇ or R₁₈; and if R₆ is not ═O, R₁₄ may be alkoxy, aryloxy, or aryl-alkoxy;
R₁ and R₂ are independently H, halide, alkoxy, alkyl, or aryl;
R₃ is H, alkyl, C₁-C₃ acyl, silyl;
R₅ is H, OH, alkyl, alkoxy, or aryloxy;
R₂₅ is alkyl, aryl, arylalkyl; and
X⁻ is an anion.

32. A composition comprising at least one compound, polymorph, or salt thereof, selected from the group consisting of: 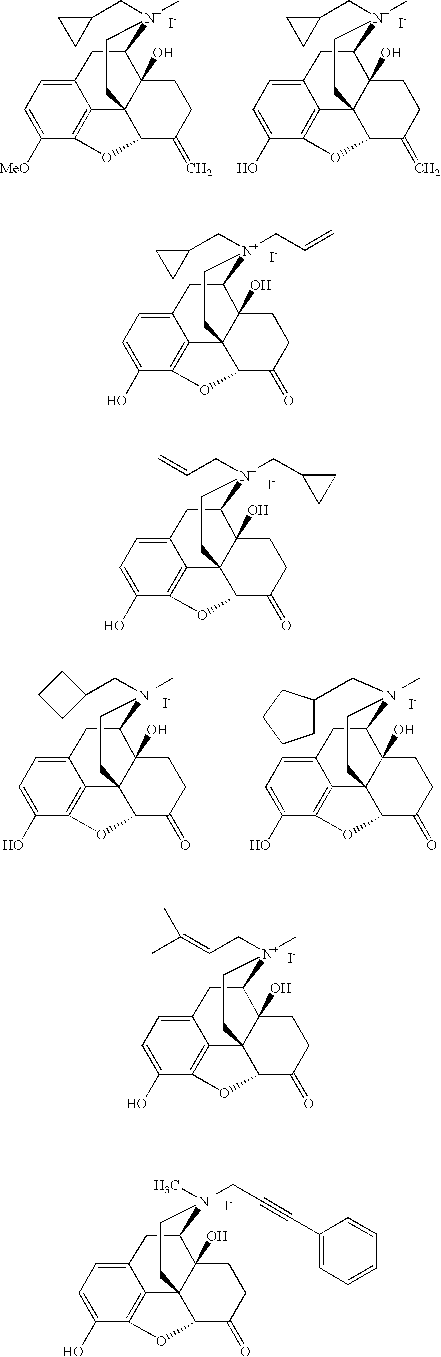 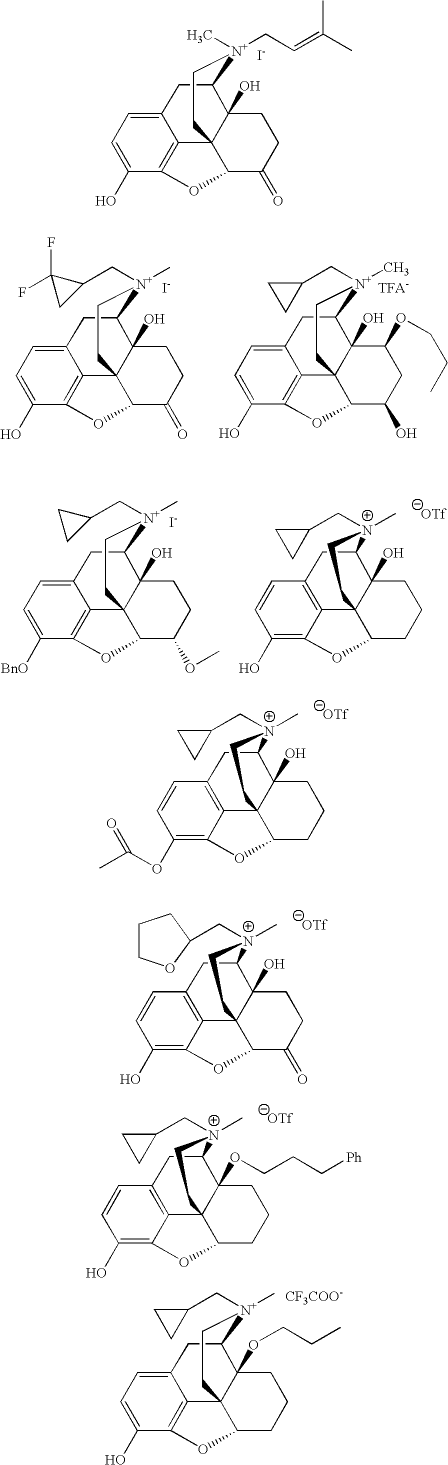 [chemical structure]
